(19) United States
(12) Patent Application Publication
(10) Pub. No.: US 2014/0249406 A1
(43) Pub. Date: Sep. 4, 2014

(54) COMPENSATOR-BASED BRACHYTHERAPY

(71) Applicant: UNIVERSITY OF IOWA RESEARCH FOUNDATION, Iowa City, IA (US)

(72) Inventors: Ryan Flynn, Iowa City, IA (US); Yusung Kim, Iowa City, IA (US); Xing Li, Iowa City, IA (US); Kee-Ho Yuen, Coralville, IA (US)

(73) Assignee: UNIVERSITY OF IOWA RESEARCH FOUNDATION, Iowa City, IA (US)

(21) Appl. No.: 14/072,270

(22) Filed: Nov. 5, 2013

Related U.S. Application Data
(63) Continuation-in-part of application No. PCT/US2012/036979, filed on May 8, 2011.
(60) Provisional application No. 61/483,702, filed on May 8, 2011.

(51) Int. Cl. A61N 5/10

(52) U.S. Cl. CPC A61N 5/1001; A61N 2005/1012
USPC 600/424; 600/8

(57) ABSTRACT

Compensator-based brachytherapy (CBT) for treatment of cancerous tumors or other pathologic tissues. CBT permits, in one aspect, increased dosage conformity for non-radially symmetric tumors by utilizing a device that can shield radiation emanated from an electronic brachytherapy (BT) source or non-electronic BT source. The device can comprise, in one aspect, a radiation compensator having a treated surface that comprises a position-dependent thickness based at least on a radiation therapy plan specific to a patient and geometry of a patient region to be treated. In an additional or alternative aspect, the device can comprise a source of radiation movably inserted into an enclosure coupled to the radiation compensator. As part of CBT, in one implementation, the radiation source can reside at a plurality of locations within the radiator compensator during a respective plurality of dwell times based on the radiation therapy plan.

### CROSS-REFERENCE TO RELATED APPLICATION

This application is a continuation-in-part of PCT Patent Application No. PCT/US2012/036979, filed on May 8, 2012, entitled “Compensator-Based Brachytherapy” which claims benefit of U.S. Provisional Patent Application No. 61/483,702 filed on May 8, 2011, entitled “Compensator-Based Intensity Modulated Brachytherapy”, the entirety of which is incorporated by references herein.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Certain embodiments of the disclosure relate to a therapeutic technique for modulation of the intensity of X-rays or gamma-rays emanating from a radiation source utilized to treat cancerous tumors. Such technique is referred to as compensator-based intensity modulated brachytherapy or compensator-based brachytherapy (CBT), and can enable treatment that is a non-invasive alternative to supplementary interstitial brachytherapy (BT) for 3D-imaging-guided brachytherapy of bulky cancerous tumors (e.g., cervical cancer tumors). The 3D imaging can be, for example, ultrasound imaging (USI), magnetic resonance imaging (MRI), computed-tomography (CT), positron emission tomography (PET), combinations thereof, or the like. In one aspect, the CBT can enable increased dosage conformity for non-symmetric tumors by utilizing a device that can shield radiation emanated from an electronic brachytherapy (BT) source or non-electronic BT source. The device can comprise, in one aspect, a radiation compensator having a treated surface that comprises a position-dependent thickness based at least on a radiation therapy plan specific to a patient and geometry of a patient region to be treated. In an additional or alternative aspect, the device can comprise a source of radiation movably inserted into an enclosure coupled to the radiation compensator. As part of CBT, in one implementation, the radiation source can reside at a plurality of locations within the radiator compensator during a respective plurality of dwell times based on the radiation therapy plan.

In one aspect, a method is provided. The method can comprise receiving data indicative of a radiation treatment and topology of a region to be treated (e.g., a volume or a surface to be treated); generating a position-dependent thickness profile of a radiation compensator surface based on the data indicative of the radiation treatment and the topology of the region to be treated; and generating a plurality of dwell times for a radiation source based on the thickness profile, wherein the radiation source is movably coupled to a radiation compensator and is adapted to reside at a plurality of locations within the radiation compensator during a respective plurality of periods, each period of the plurality of periods being equal to a respective dwell time of the plurality of dwell times. In certain embodiments, the method can further comprise supplying a treatment plan comprising the position-dependent thickness profile and the plurality of dwell times, wherein generating a position-dependent thickness profile of a radiation compensator surface based on the data indicative of the radiation treatment and the topology of the region to be treated can comprise discretizing the radiation compensator surface into a plurality of voxels and assigning a respective initial plurality of thicknesses to the plurality of voxels; and determining an extremum of an objective function by iteratively updating each thickness of the respective initial plurality of thicknesses and each dwell time of an initial plurality of dwell times, wherein the objective function is indicative of a difference among a prescribed dose at a position in the region to be treated and an actual dose provided at the position, the updating step yielding a current plurality of thicknesses and a current plurality of dwell times. In one aspect, the method, in response to identifying the extremum, can comprise performing the steps of configuring the current plurality of thicknesses as the position-dependent thickness profile; and configuring the current plurality of dwell times as the plurality of dwell times.

In another aspect, a computer-readable storage medium encoded with computer-executable instructions is provided. The computer-executable instructions can comprise first computer-executable instructions that, in response to execution, cause a processor to receive data indicative of a radiation treatment and topology of an area to be treated; second computer-executable instructions that, in response to execution, cause the processor to generate a position-dependent thickness profile of a radiation compensator surface based on the data indicative of the radiation treatment and the topology of the region to be treated; and third computer-executable instructions that, in response to execution, cause the processor to generate a plurality of dwell times for a radiation source based on the thickness profile, wherein the radiation source is movably coupled to a radiation compensator and is adapted to reside at a plurality of locations within the radiation compensator during a respective plurality of periods, each period of the plurality of periods being equal to a respective dwell time of the plurality of dwell times.

In yet another aspect, a device is provided. The device can comprise a radiation compensator having a treated surface having a position-dependent thickness according to a thickness profile based on a radiation therapy plan and geometry of a region to be treated; and a source of radiation movably inserted into a first enclosure coupled to the radiation compensator, wherein the radiation source is adapted to reside at a plurality of locations within the radiation compensator during a respective plurality of periods, each period of the plurality of periods being equal to a respective dwell time of the plurality of dwell times, and wherein each dwell time is based on the radiation therapy plan. In certain embodiments, the radiation compensator resides within a second enclosure that encompasses the first enclosure, the first enclosure adapted to move relative to the second enclosure, and wherein the second enclosure is coupled to alignment means for positioning the first enclosure relative to the second enclosure. In other embodiments, the alignment means for positioning the first enclosure relative to the second enclosure comprises means for indicating orientation of the second enclosure relative to the region to be treated; and means for locking at least part of the first enclosure outside the second enclosure in response to misalignment between orientation of the first enclosure and the orientation of the second enclosure. In certain embodiments, the means for indicating orientation of the second enclosure relative to the region to be treated are adapted to be visible on an three-dimensional imaging system.

Additional aspects, features, or advantages of the subject disclosure will be set forth in part in the description which follows, and in part will be obvious from the description, or may be learned by practice of the subject disclosure. The advantages of the subject disclosure will be realized and attained by means of the elements and combinations particularly pointed out in the appended claims. It is to be understood that both the foregoing general description and the following detailed description are exemplary and explanatory only and are not restrictive of the subject disclosure.

### BRIEF DESCRIPTION OF THE DRAWINGS

The accompanying drawings are incorporated and illustrate exemplary embodiment(s) of the disclosure and together with the description and claims appended hereto serve to explain various principles, features, or aspects of the subject disclosure.

FIG. 1A illustrates conventional BT with ¹⁹²Ir combined with external beam radiation therapy (EBRT): D₉₀ for the high-risk clinical target volume (HR-CTV) is restricted to 64 Gy_{EQD2} due to the requirement that no more than 2 cm³ of contiguous bladder, rectum, or sigmoid can receive doses greater than 90 Gy_{EQD2}, 75 Gy_{EQD2}, and 75 Gy_{EQD2}, respectively, in accordance with GEC-ESTRO recommendations. D₉₀ is the maximum dose delivered to the hottest 90% of a volume. The radiation dose in units of Gy_{EQD2} is the total dose delivered when delivered in 2 Gy fractions. FIG. 1B illustrates an exemplary conventional brachytherapy (BT) dose distribution. FIG. 1C illustrates an exemplary CBT combined with EBRT dose distribution in accordance with aspects of the disclosure. Such dose distribution satisfies the same bladder, rectum, and sigmoid sparing requirements as in FIG. 1A, but for which D₉₀ for the HR-CTV is 90 GyEQD2.

FIG. 2 illustrates an example CBT delivery scheme in accordance with one or more aspects of the disclosure.

FIG. 3 illustrates a cross sectional view of an IMBT insertion device in accordance with aspects described herein.

FIG. 4 illustrates exemplary resulting tumor surface dose distributions for radiation treatment of a tumor with conventional BT (shown in panel (a)) and according to CBT as described herein.

FIG. 5 illustrates exemplary dose-surface histograms for tumor of FIG. 5.

FIG. 6 illustrates computed (e.g., optimized) dwell times on a relative scale for the various source positions in an applicator, or insertion device, for both conventional BT and CBT as described herein.

FIGS. 7A-7B illustrates exemplary thicknesses of a radiation compensator surface in accordance with aspects described herein.

FIG. 8 illustrates dose-volume histograms for the organs depicted in FIG. 1A-IC in accordance with aspects of the subject disclosure. HR-CTV doses were limited by the bladder dose constraint (90 Gy_{EQD2} to 2 cc) for ¹⁹²Ir-based BT and eBT, and the sigmoid dose constraint (90 Gy_{EQD2} to 2 cc) for the CBT case. D₉₀ for ¹⁹²Ir, eBT, and CBT was 64, 62 and 90 Gy_{EQD2}, respectively.

FIG. 9 illustrates relative dwell times for the three techniques for different radiation treatment: BT, eBT, and CBT.

FIG. 10 illustrates a thickness profile (which also can be referred to as a distribution profile) of tungsten attenuator on the optimized compensator used to generate the dose distribution depicted in FIG. 1C.

FIGS. 11A-11B illustrate example values of thicknesses of radiation compensator formed from different materials and for various BT sources in accordance with one or more aspects of the disclosure.

FIG. 12A-12B illustrate exemplary embodiments of an apparatus for producing a radiation compensator in accordance with aspects of the subject disclosure.

FIG. 13 illustrates exemplary embodiments of an apparatus for producing a radiation compensator in accordance with one or more aspects of the disclosure.

FIG. 14 illustrates an example embodiment of an assembly to produce a compensator for CBT in accordance with one or more aspects of the disclosure.

FIG. 15 illustrates a portion of a compensator in accordance with one or more aspects of the disclosure.

FIG. 16 illustrates a device for producing a laminated compensator according to one or more aspects of the disclosure.

FIG. 17 depicts an example embodiment of a milling apparatus in accordance with one or more aspects of the disclosure.

FIG. 18 depicts an example radiopaque material and an example radiation compensator in accordance with one or more aspects of the disclosure.

FIG. 19 illustrates an example milling procedure in accordance with one or more aspects of the subject disclosure.

FIG. 20 illustrates a cross-section of an example phantom in accordance with one or more aspects of the disclosure.

FIG. 21 illustrates an exemplary embodiment of an applicator having alignment means for aligning a radiation compensator and the application in accordance with one or more aspects of the disclosure.

FIG. 22 is a flowchart of an exemplary method for providing a radiation compensator in accordance with one or more aspects of the disclosure.

FIG. 23 is a flowchart of an exemplary method for conducting therapeutic treatment with a medical device for implementing radiation therapy in accordance with aspects described herein.

FIG. 24 illustrates a computing environment that enables various aspects of compensator design and/or automation of compensator fabrication in accordance with aspects described herein.

FIGS. 25-27 illustrate a curved CBT applicator system according to an aspect of the present invention.

### DETAILED DESCRIPTION

The subject disclosure may be understood more readily by reference to the following detailed description of exemplary embodiments of the subject disclosure and to the Figures and their previous and following description.

Before the present compounds, compositions, articles, devices, and/or methods are disclosed and described, it is to be understood that the subject disclosure is not limited to specific systems and methods for compensator-based brachytherapy and related devices. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

In the subject specification and in the claims which follow, reference may be made to a number of terms which shall be defined to have the following meanings: “Optional” or “optionally” means that the subsequently described event or circumstance may or may not occur, and that the description includes instances where said event or circumstance occurs and instances where it does not.

As employed in this specification and annexed drawings, the terms “unit,” “component,” “interface,” “system,” “platform,” “stage,” and the like are intended to include a computer-related entity or an entity related to an operational apparatus with one or more specific functionalities, wherein the computer-related entity or the entity related to the operational apparatus can be either hardware, a combination of hardware and software, software, or software in execution. One or more of such entities are also referred to as “functional elements.” As an example, a unit may be, but is not limited to being, a process running on a processor, a processor, an object, an executable computer program, a thread of execution, a program, a memory (e.g., a hard disc drive), and/or a computer. As another example, a unit can be an apparatus with specific functionality provided by mechanical parts operated by electric or electronic circuitry which is operated by a software or a firmware application executed by a processor, wherein the processor can be internal or external to the apparatus and executes at least a part of the software or firmware application. In addition or in the alternative, a unit can provide specific functionality based on physical structure or specific arrangement of hardware elements. As yet another example, a unit can be an apparatus that provides specific functionality through electronic functional elements without mechanical parts, the electronic functional elements can include a processor therein to execute software or firmware that provides at least in part the functionality of the electronic functional elements. An illustration of such apparatus can be control circuitry, such as a programmable logic controller. The foregoing example and related illustrations are but a few examples and are not intended to be limiting. Moreover, while such illustrations are presented for a unit, the foregoing examples also apply to a component, a system, a platform, and the like. It is noted that in certain embodiments, or in connection with certain aspects or features thereof, the terms “unit,” “component,” “system,” “interface,” “platform” can be utilized interchangeably.

Throughout the description and claims of this specification, the words “comprise,” “include,” and “have” and variations of the word, such as “comprising,” “comprises,” “including,” “includes,” “has,” and “having” mean “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Reference will now be made in detail to the various embodiment(s), aspects, and features of the subject disclosure, example(s) of which are illustrated in the accompanying drawings. Wherever possible, the same reference numbers are used throughout the drawings to refer to the same or like parts.

As described in greater detail below, the disclosure relates, in one aspect, to a therapeutic technique for modulation the intensity of X-rays or gamma-rays emanating from a radiation source utilized to treat cancerous tumors. Such technique can be referred to as CBT and enables treatment that is a non-invasive alternative to supplementary interstitial brachytherapy (BT) for 3D-imaging-guided brachytherapy of bulky cancerous tumors, such as cervical tumors. The 3D imaging can be, for example, USI, MRI, PET, and/or CT. In one aspect, CBT dosage distributions can be generated by isotopes such as ¹⁹²Ir, ¹³¹Cs, ¹²⁵I, ¹⁰³Pd, ¹⁹⁸Au, ¹⁸⁷W, ¹⁶⁹Yb, ¹⁴⁵Sm, ¹³⁷Cs, ¹⁰⁹Cd, ⁶⁵Zn, ¹⁵³Gd, ⁵⁷Co, ⁵⁶Co, and ⁵⁸Co, or an electronic BT (eBT) source wrapped or otherwise contained in a novel compensator that is coated with varying thicknesses of high-Z material (e.g., atomic number Z greater than or equal to 22). Such isotopes can be referred to as, for example, non-electronic BT sources. In another aspect, CBT can permit treatment of lateral tumor extensions to dosages that are unlikely—and even unfeasible—to be delivered with conventional intracavitary BT due to dose limitations that can be imposed by presence of nearby healthy tissue (such as the bladder, rectum, and sigmoid in case of cervical cancer treatment). In another aspect, CBT can enable increased dosage conformity for non-symmetric tumors by utilizing a device that can shield radiation emanated from an electronic brachytherapy (BT) source or non-electronic BT source. The device can comprise, in one aspect, a radiation compensator having a treated surface that comprises a position-dependent thickness based at least on a radiation therapy plan specific to a patient and geometry of a patient region to be treated. In an additional or alternative aspect, the device can comprise a source of radiation movably inserted into an enclosure coupled to the radiation compensator. As part of CBT, in one implementation, the radiation source can reside at a plurality of locations within the radiator compensator during a respective plurality of dwell times based on the radiation therapy plan.

Various aspects or features of the disclosure can be applied to the field of radiation oncology. Conventional brachytherapy entails the insertion of radioactive sources into tumors through interstitial needles or intracavitary applicators, and delivers very high radiation doses to tumors but often with poor tumor dose conformity. Without wishing to be bound by theory and/or simulation, such poor tumor dose conformity is due to the fact that conventional BT dose distributions typically are radially symmetric and tumors usually are not. It should be appreciated that poor dose conformity is of clinical concern since tumor underdosage leads to recurrence and tumor overdosage excessively damages nearby healthy tissue. One or more embodiments of the disclosure can rectify such deficiency by wrapping the BT source with a patient-specific treatment-specific compensator that can be covered with spatially-varying (or position dependent) thicknesses of an attenuating material, e.g., a metal with high atomic number, such as lead, iron, gold, et cetera. In certain embodiments, the radiation compensator thickness distribution, or radiation compensator thickness profile, can be optimized or nearly optimized through computations based on the BT source positions in the tumor, tumor shape, and/or the desired radiation dose distribution associated with specific radiation treatment. In other embodiments, the radiation compensator thickness distribution can be designed to satisfy certain criteria not necessarily comprising optimization, but rather achieving a desired performance of a medical device for radiation treatment that employs the radiation compensator. Poor dose conformity can be prevented by shielding regions that would be conventionally overdosed more than regions that would be conventionally underdosed. In certain embodiments, radiation compensators can be fabricated by printing attenuating material on bendable plastic substrates, laminating, and/or wrapping the compensator around the source of treatment. In other embodiments, a radiation compensator can be produced by milling a surface of a radiopaque material according to a predetermined thickness profile. In yet other embodiments, radiation compensators can be generated by the milling cavities, or pockets, of a surface of a slab of solid material, filling at least a portion of the cavities with a radiopaque material, and laminating the resulting milled surface to yield an flexible compensator. In one aspect, CBT can be of commercial value because it is a feasible treatment that can provide improvement over conventional BT and can result in improved patient care. Examples of cancers that can be treated more effectively with CBT comprise vaginal, cervical, endometrial, breast, lung, liver/bile duct, and/or prostate tumors.

One or more of the principles of the disclosure can be utilized in various therapeutic radiation treatments. In one aspect, an exemplary application of CBT is in the field of radiation oncology. More specifically, yet not exclusively, CBT can be utilized for the treatment of tumors that are not radially symmetric about certain axis. In one example, CBT can overcome one or more limiting factors of treating breast lumpectomy cavities. In one embodiment, an electronic brachytherapy source, such as the Xoft (Sunnyvale, Calif.) Axxent™ can be inserted through a catheter and into a saline-filled balloon having a radius from about 1 cm to about 2 cm and being located inside the breast in order to treat the tissue within 5 mm of the balloon surface. In CBT as described herein, BT sources are not limited to electronic brachytherapy sources. The dose received by the target tissue can be sufficiently sensitive to the balloon shape that the procedure may be aborted due to slight defects (e.g., distortion of about 2 mm) in the radial symmetry of the balloon, if balloon-to-skin distance is less than 7 mm, and/or if non-conforming air or seroma is present in the cavity. Cancellation of treatment generally requires that the patient return on a different day for re-setup and re-imaging, which can be time-consuming and expensive. In one aspect, CBT can enable the delivery of dose distributions that overcome such limitations, removing the need to cancel treatment. Another example of a problem that CBT can overcome is the treatment of cervical cancer tumors, which rarely are radially symmetric. In one embodiment, CBT can deliver doses to cervical cancer tumors that are impractical to deliver with conventional BT.

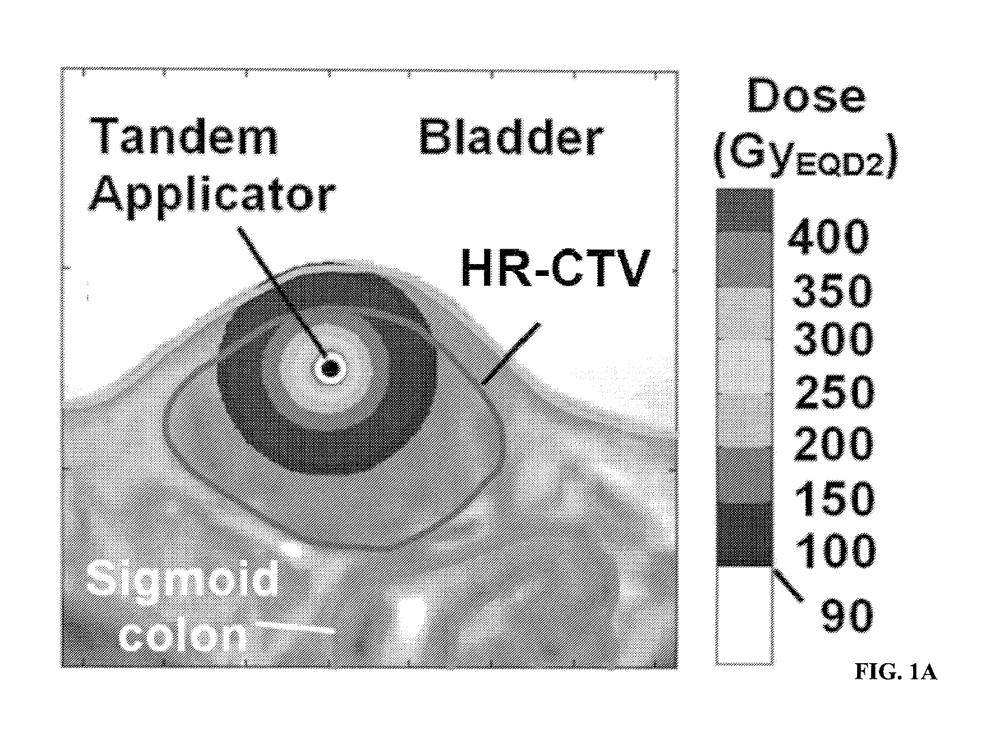

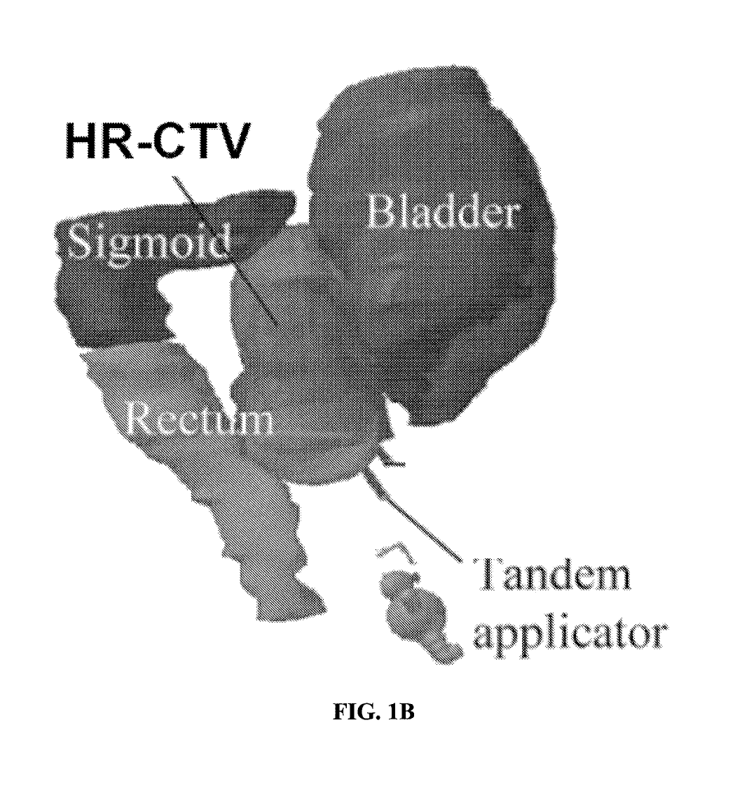

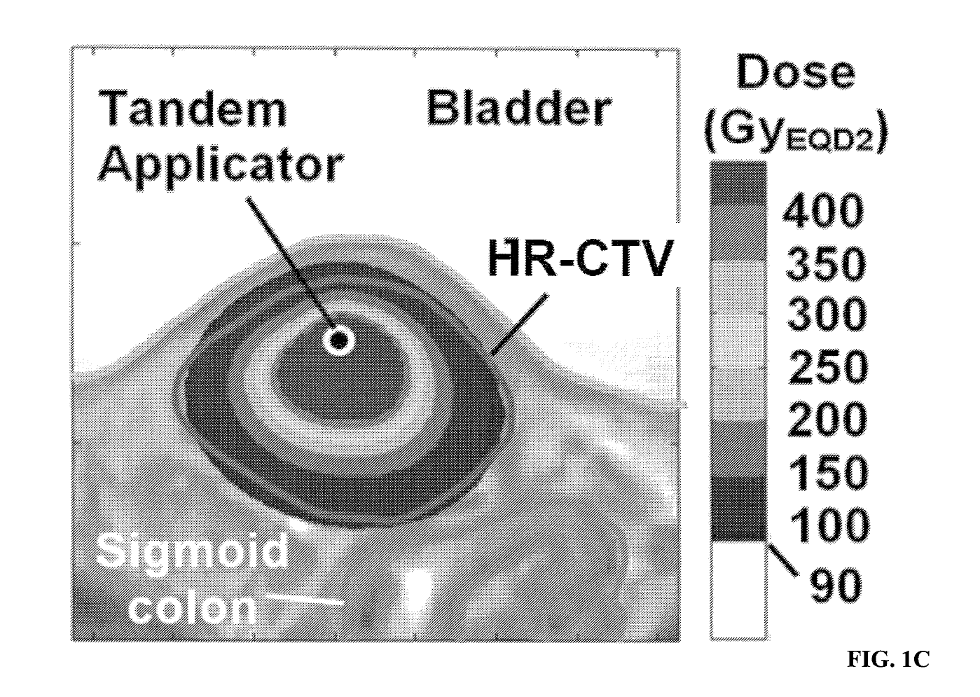

Brachytherapy, or “short-distance therapy,” treats target tissues, such as cancerous tumors, with radiation sources that can be placed inside or directly adjacent to the target tissue using some applicator. Example target tissues include cervical, vaginal, endometrial, breast, and skin cancers. Example applicators include interstitial needles and intracavitary applicators. The advantage of brachytherapy over EBRT is that EBRT beams usually must pass through healthy tissue in order to reach their targets, while the radiation used in brachytherapy may not. As a result brachytherapy can be used to treat targets with very high radiation doses relative to those achievable with EBRT, with less concern for overdosing nearby healthy tissue. The application of 3-D imaging systems such as USI, CT, and MRI for brachytherapy guidance has revealed that the dose conformity to tumors is often poor. Without wishing to be bound by theory and/or simulation, it is believed that poor conformity of conventional brachytherapy (BT) typically is delivered with isotopes or electronic sources that emit radiation in a radially symmetric manner, yet tumors often are not radially symmetric. For example, FIG. 1B illustrates MRI-generated 3D renderings of the anatomy of a patient being treated for cervical cancer, including the tumor and nearby critical structures: bladder, rectum, and sigmoid colon. The radiation is delivered with an X-ray or gamma-ray emitting source that travels through a set of rigid tandem and ovoid (T&O) applicators inserted into the anesthetized patient. The radially symmetric dose distribution emitted by conventional BT sources, however, results in the poor tumor coverage as shown in FIG. 1B. The desired radiation dose to the tumor, shown as the red outline, is 100% of the prescribed radiation dose, which is clearly not being achieved in a large fraction of the tumor. Improved tumor coverage can be achieved with intensity modulated brachytherapy (IMBT), which uses shielding of the radiation source to achieve a better dose distribution. Improved tumor coverage obtained with IMBT can be expected to increase local tumor control probability in any applicable tumor, improving patient outcomes.

The feasibility of IMBT has been investigated and it has been demonstrated that IMBT could be delivered using radioisotopes and the Xoft (Sunnyvale, Calif.) Axxent electronic brachytherapy source, respectively, by collimating the source with high-density shields that create fan beams. The fan beam source is rotated inside the patient in a manner such that the amount of time the source spends irradiating a given direction is optimized to ensure better tumor coverage and better critical structure avoidance than conventional brachytherapy. Although both approaches support the potential benefits of IMBT, there are two major challenges associated with the rotating shield approach to IMBT delivery. First, rotating and verifying the location of a moving shield inside a curved applicator is non-trivial. Second, the delivery times associated with IMBT are increased relative to conventional BT. This is due to the loss of emitted radiation in the rotating shield, which must remove a large fraction, possibly around 90%, of the radiation in order to achieve an advantage over conventional BT. If the rotating fan beam accounts for only 10% of the radiation emitted by the BT source and the rest is lost in the shield, then delivering the same dose distribution as conventional BT will require at least ten times as long with rotating-shield IMBT. This is because the fan will have to be pointed in 10 directions and stay pointed in each direction for the same amount of time necessary to deliver an entire conventional BT plan, which loses 0% of the radiation due to shielding.

In another aspect, of the nearly 11,000 annual cases of newly-diagnosed cervical cancer in the U.S., about 45% (5,000) are of stage IB2 or higher. Cervical cancer of stage IB2 or higher has 5-year survival rates of up to about 70%, and 5-year survival and local control ranges from 0-20% and 18-48%, respectively, for stage IVA tumors. Such cancers typically are treated with a combination of chemotherapy, external beam radiation therapy (EBRT), and an intracavitary BT boost to the tumor. The advent of MRI-guided BT has revealed that the close proximity of the bladder, rectum, and sigmoid to the tumor restrict the radiation dose that can be delivered to the non-symmetric extensions of bulky (e.g., greater than about 40 cc) tumors with conventional BT, likely reducing the chances of local control. Tumor dose conformity for such bulky tumors can be significantly improved through the use of supplementary BT through interstitial needles, which is more invasive than intracavitary BT, may cause complications, and can add 35-70 minutes to the BT procedure.

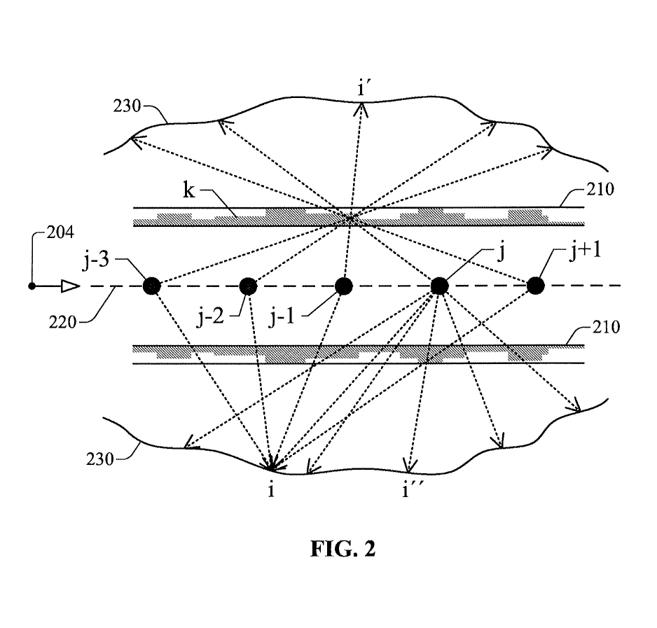

The radially symmetric dose distributions of conventional BT poorly conform to non-symmetric cervical cancer tumors, an example of which is illustrated in FIG. 1A. Conventional BT is delivered to tumors with an ¹⁹²Ir (380 keV average energy) radiation source while traveling through rigid intracavitary applicators. To deliver CBT, slightly modified applicators can be utilized, but with introduction of a radiation compensator having a treated surface in accordance with one or more aspects described herein. In one aspect, the radiation compensator can be coated with a high-density attenuating material (e.g., gold or titanium) having varying thicknesses, and can be wrapped around the eBT source. In certain scenarios, a single radiation compensator can be employed for a CBT treatment, and multiple dwell positions can be utilized (see, e.g., FIG. 2). In one aspect, the radiation compensator can regulate the radiation intensity emitted in all directions or most all directions, enabling the treatment, for example, of non-symmetric tumors by preventing sensitive structures from restricting tumor dose as shown in FIG. 1C. As described herein, CBT can produce substantial tumor dose conformity gains relative to conventional BT at clinically feasible treatment times without violating the GEC-ESTRO recommended bladder, rectum, and sigmoid doses.

Compensator-based IMBT is a process for delivering IMBT with no moving parts in addition to those already present for conventional BT. In one aspect, with CBT, a source-containing catheter that is inserted into an applicator or the source itself wrapped in a patient-specific compensator that is covered with space-dependent thicknesses of an attenuating material, such as titanium, tungsten, or lead. The distribution of thicknesses of the attenuating material forming, in part, the radiation compensator surface can be determined by computerized optimization incorporating data indicative of tumor shape and applicator shape. At least a portion of such data can be obtained via an imaging technique, such as MRI, CT, or the like. As an example, FIGS. 1A-1B illustrate cross-sectional images of a tumor and surrounding tissues. The distribution of thicknesses of the attenuating material contained in the radiation compensator surface can be referred to as a thickness profile of the radiation compensator surface.

Certain principles of CIBT in accordance with the disclosure are illustrated in FIG. 2, which depict brachytherapy source positions (represented with solid dots in the drawings) and radiation transport patterns on a plane containing the axis along which the BT source, or radiation source, moves through an applicator 210 (or insertion device), as illustrated in a cross-sectional view in FIG. 2. In general, the brachytherapy source is inserted into the applicator 210, or the insertion device, and allowed to dwell for respective dwell time intervals tⱼ at one or more positions, each of the one or more positions being indexed by an integer j along the applicator axis 220. Such positions referred to as dwell positions. In one aspect, the brachytherapy source can be inserted into a catheter that fits within the applicator, or insertion device. It should be appreciated that radiation can be emitted by the BT source in all directions or substantially all directions from each dwell position. A region on the compensator, indexed by the integer k, of physical thickness ηₖ (which can be a thickness of the order of a μm) affects the radiation dose delivered at multiple points in the tumor. Such regions are illustrated in FIG. 2 with grey blocks. In FIG. 2, arrows depict radiation transport lines starting at the dwell positions, passing through compensator element k, and reaching the tumor surface 230. A given voxel in the tumor, indexed by the integer i, can be affected by radiation arising from multiple combinations of BT sources at different dwell positions (e.g. j−3, j−2, j−1, j, j+1, and j+2) and locations in the compensator, as shown in FIG. 2 by arrows that start at the dwell positions (represented with solid dots) and pass through different compensator elements while propagating towards tumor voxel i. In one aspect, voxel i can receive a radiation dose dᵢ. Voxels i′ and i″ also are illustrated in FIG. 2.

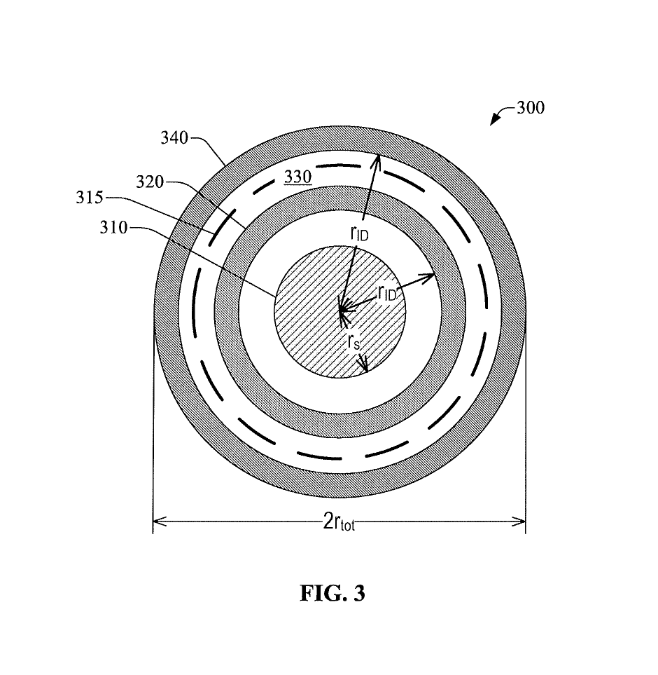

A cross sectional view of a CBT insertion device 300 is illustrated in FIG. 3, which depicts the relative locations of the BT source 310, a catheter tube 320 (or catheter 320), a space 330 for a radiation compensator in accordance with one or more aspects described herein, and applicator 340. The CBT insertion device 300 be embodied in a needle or an intracavitary applicator of inner radius r_{ID} and outer radius rₜₒₜ. In one aspect, the radiation source 310 can have an outer radius rₛ. The radiation source 310 can move through the catheter tube 320, which can have an outer radius r_{c}. In one aspect, the catheter tube 320 forms, at least in part, a first enclosure into which the radiation source 310 can be inserted. In embodiments, such as the illustrated embodiment, in which a radiation compensator 315 (indicated with a thick dashed line) fits in the space 330 between the catheter tube 320 and the applicator 340, the CBT of the disclosure is feasible and can be implemented. More generally, the CBT can be implemented in embodiment in which ample or sufficient space exists in the CBT insertion device 300, between r_{c} and r_{ID} for insertion of the radiation compensator. It is noted that CBT also can be implemented in embodiments in which no catheter tube 320 is used between the BT source 310 and the inner surface of the applicator 340.

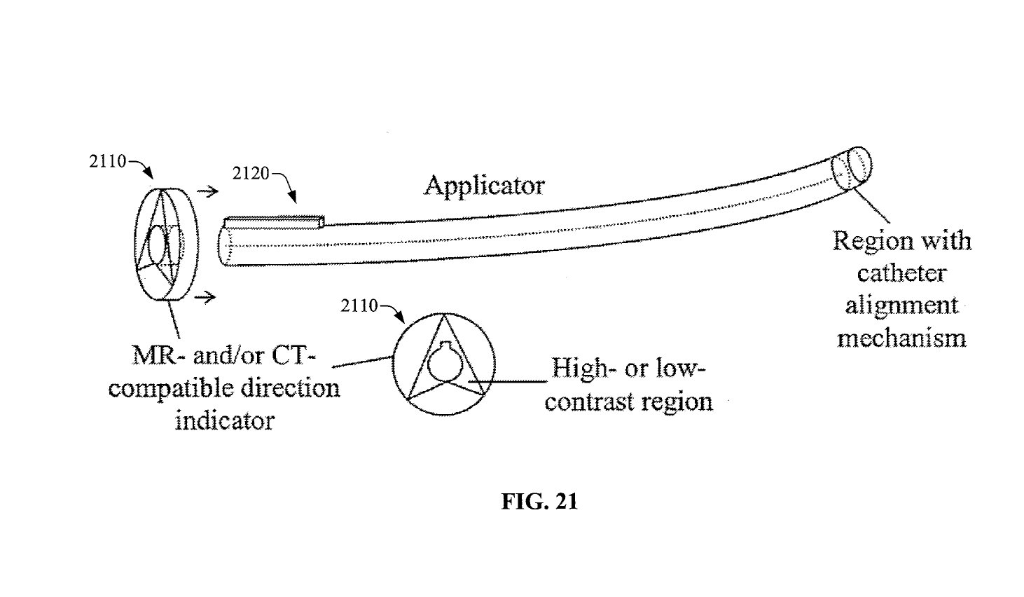

It should be appreciated that the radiation compensator is coupled to the first enclosure formed by the catheter tube 320. In another aspect, the space 330 is bound by the applicator 340 (see, e.g., FIG. 21) and the catheter tube 320 and forms a second enclosure that encompasses the first enclosure. As described herein, the catheter 320, which can form the first enclosure can be adapted to move relative to the second enclosure, defined in part by the applicator 340. As described herein, in one embodiment, the applicator 340 and thus the second enclosure can be coupled to alignment means for positioning the first enclosure (e.g., the catheter tube 320) relative to the second enclosure (e.g., the applicator 340).

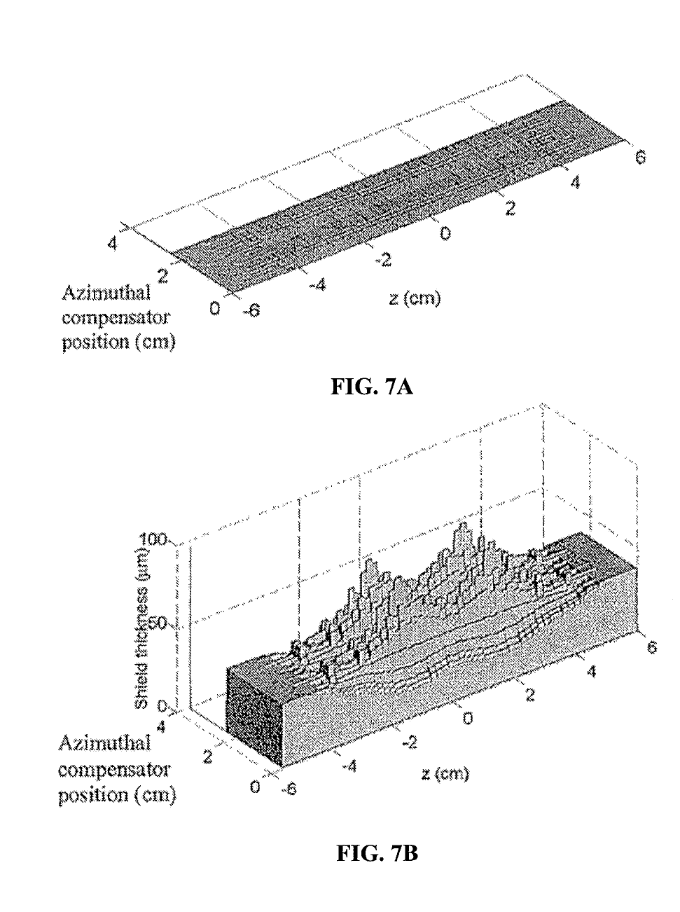

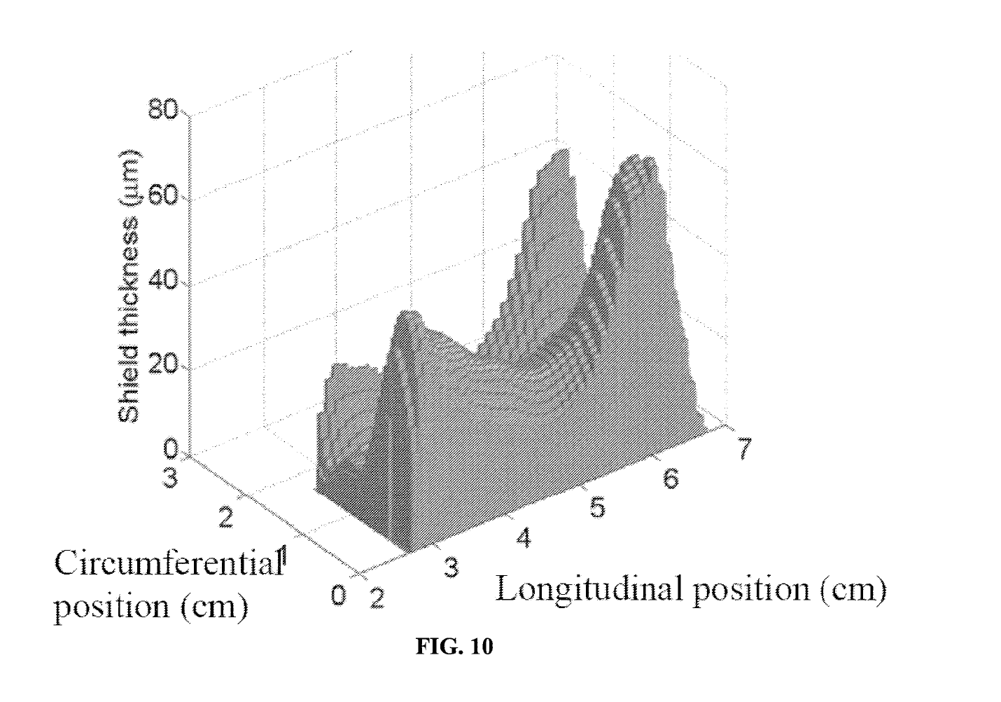

In one aspect, as described herein, the radiation compensator 315 has a treated surface (e.g., milled, sputtered, etched, printed, sintered, laminated, or any combination thereof) having a position-dependent thickness according to a thickness profile, such as the thickness profile of FIG. 7B or FIG. 10. As described herein, the thickness profile can be based on a radiation therapy plan and geometry of a region to be treated.

During CBT, in one aspect, as described herein, the radiation source can be adapted (e.g., sized and mounted to displacement means) to reside at a plurality of locations within the radiation compensator 315 during a respective plurality of periods, each period of the plurality of periods being equal to a respective dwell time of the plurality of dwell times, and wherein each dwell time is based on the radiation therapy plan.

As illustrated in FIG. 2, the radiation source 204 (or brachytherapy source, indicated with a small solid dot) can be displaced (indicated with an open-head arrow attached to the radiation source 204) inside the applicator 210 from left to right, from example, stopping at the dwell position indexed by j (a natural number) to emit radiation for a predetermined dwell time tⱼ. Each compensator attenuation element indexed by k has a thickness ηₖ and affects radiation dose delivered at many points on the tumor surface. Similarly, the radiation dosage dᵢ at an arbitrary tumor voxel i can be affected by all dwell positions and multiple attenuation element combinations.

In one aspect, implementation of CBT can comprise determination of optimal radiation compensator thicknesses for a specific target shape (e.g., tumor shape or shape of a region to be treated) and radiation dosage prescription. It may not be readily apparent that wrapping or otherwise covering the radiation source or catheter tube with a compensator can result in a significant advantage over conventional BT, especially yet not exclusively for the case of a treatment delivered using multiple dwell positions. Provided that IMBT delivery using a shield that rotates about the radiation source at each dwell position is part of conventional technology, a compensator that remains stationary throughout the delivery or radiation, or treatment, may appear to provide a limited amount of freedom to modulate the radiation source emissions in an advantageous manner. Yet, through computational modeling as described herein, in one aspect, it can be demonstrated that it is possible to customize (optimally, non-optimally, or according to a predetermined criterion) the radiation compensator thickness distribution (or radiation compensator thickness profile) in a manner that provides an advantage over conventional BT without the complication of additional moving parts associated with rotating-shield IMBT.

The total radiation dose delivered to voxel i from the radiation source with CBT can be approximated, in one aspect, as:

$${d}_{i} = {\sum }_{j} {\dot{D}}_{ij} {t}_{j} {T}_{Δ x}^{{η}_{{k}_{ij}} / Δ x cos {θ}_{ij}} , \qquad Eq . ( 1 )$$

wherein \dot{D}ᵢⱼ is the dose rate at tumor voxel i due to source emissions at dwell position j and tⱼ is the dwell time at source position j; T_{Δx} is the source-dependent reference radiation transmission factor for a ray passing through the specific compensator material, such as a radiopaque material of high atomic number Z, (e.g., 78, 79), or an alloy of two or more such radiopaque materials, having a reference thickness Δx, which can be configured to a specific value (e.g., about 100 μm). The reference radiation transmission factor can be calculated, in one aspect, as follows:

$${T}_{Δ x} = \frac{{\int }_{0}^{∞} d E f ( E ) {e}^{- μ ( E ) Δ x}}{{\int }_{0}^{∞} d E f ( E )} , \qquad Eq . ( 2 )$$

where ƒ(E) is a real function describing the emission per unit energy of the radiation source for energy E. For example, ƒ(E) can be the fluence spectrum Φ′(E), which is measured in units of photons cm⁻² MeV⁻¹, or the energy fluence spectrum, Ψ′(E)=EΦ′(E) which is measured in units of cm⁻² of the radiation source. Here, μ(E) is an energy-dependent absorption coefficient which can be determined as the product between a mass energy absorption coefficient μ(E)/ρ (in units of cm²/g, for example) and the density p (in units of g/cm³, for example) of a medium in which radiation is propagated: Raising T_{Δx} to the power of ηₖ_{ᵢⱼ}/Δx cos θᵢⱼ yields the compensator transmission along the radiation transport line that begins at dwell position j and ends at voxel i. The k-subscript of ηₖ, the compensator thickness distribution, includes the “ij” subscript because a specific ηₖ element can affect multiple voxels and source position pairs, as shown in respective top portions of FIG. 2. Thus, for a specific composite index ij, a suitable k-subscript of η is identified in order to determine the attenuation between source position j and tumor voxel i. Angle θᵢⱼ is defined as the angle of incidence of the radiation transport line ij on the radiation compensator surface, with θ=0 corresponding to normal incidence. Here, ηₖ_{ᵢⱼ} is divided by Δx cos θᵢⱼ in Eq. (1) to account for the reference attenuator thickness of Δx and possible pathlength increase due to oblique incidence of radiation transport line with the radiation compensator.

In one aspect, the central computational problem of CBT comprises finding a satisfactory (optimal, nearly-optimal, etc.) vector of dwell times \vec{t}′ and an optimal vector of compensator thicknesses (or thickness profile) \vec{η}′ that produce a dose vector \vec{d}(\vec{t}′, \vec{η}′) that minimizes the magnitude of the difference vector \vec{δ}=\vec{d}(\vec{t}′, \vec{η}′)−\vec{d}^{(p)} or yields a magnitude of value \vec{δ} within a predetermined tolerance δ₀ (a real value), wherein \vec{d}^{(p)} is a prescribed radiation dose vector. As described herein, in addition to the magnitude of \vec{δ}, other objective functions that quantify agreement between \vec{d}^{(p)} and \vec{d}(\vec{t}′, \vec{η}′) can be utilized. For an available thickness profile \vec{η}′, a radiation compensator with a customized thickness according to such thickness profile can be manufactured through various processes in accordance with aspects described herein. A manufactured radiation compensator having the thickness profile \vec{η}′ can be inserted into an applicator, or CBT insertion device and the radiation treatment can be delivered using the satisfactory (e.g., optimized) dwell times. In one aspect, the manufactured radiation compensator can be inserted into the applicator by wrapping or otherwise mounting the compensator around the radiation source. In another aspect, in scenarios in which a catheter is available, the manufactured radiation compensator can be wrapped around the catheter in order to insert the compensator into the applicator.

In certain embodiments, vectors and \vec{t}′ and \vec{η}′ can be determined by computer-based stochastic optimization or deterministic optimization, which typically can involve, as described herein, determining an extremum of an objective function that quantifies the agreement between \vec{d}^{(p)} and \vec{d}(\vec{t}′, \vec{η}′). In one aspect, a maximum of the objective function can be determined. In another aspect, a minimum of the objective function can be determined. It should be appreciated that many of the optimization algorithms that can be employed to determine an extremum of the objective function can benefit from an analytical expression for the gradient of the objective function with respect to one or more optimization parameters. As an example, in embodiments in which the objective function is F[\vec{d}(\vec{t}′, \vec{η}′)], the elements of the gradient of F can be obtained, in general, according to the following equations:

$$\begin{matrix}\frac{\partial F}{\partial {t}_{j}} = {\sum }_{i} \frac{\partial F}{\partial {d}_{i}} \frac{\partial F}{\partial {t}_{j}} \\ and & Eq . ( 3 ) \\ \frac{\partial F}{\partial {η}_{k}} = {\sum }_{i} \frac{\partial F}{\partial {d}_{i}} \frac{\partial F}{\partial {η}_{k}} & Eq . ( 4 )\end{matrix}$$

Based on Eq. (1), the following is obtained:

$$\frac{\partial {d}_{i}}{\partial {t}_{j}} = {\dot{D}}_{ij} {T}_{Δ x}^{{η}_{{k}_{ij}} / Δ x cos {θ}_{ij}}$$

for all indices i, and

$$\frac{\partial {d}_{i}}{\partial {η}_{k }} = \{ \begin{matrix}{\sum }_{j} {\dot{D}}_{ij} {t}_{j} \frac{ln {T}_{Δ x}}{Δ x cos {θ}_{ij}} {T}_{Δ x}^{{η}_{{k}_{ij}} / Δ x cos {θ}_{ij}} for i ∈ {I}_{k} \\ 0 otherwise .\end{matrix}$$

In the foregoing, Iₖ is the set of one or more voxel indices i that are affected by radiation compensator element k and a dwell position j, as illustrated in FIG. 2. Thus, in certain implementations, gradient-based optimization methods can be utilized to generate BT and CBT treatment plans by minimizing the objective function F[\vec{d}(\vec{t}′, \vec{η}′)].

In certain embodiments, the objective function can be a quadratic objective function, such as

$$F [ {d}^{->} ( {{t}^{->}}^{′} , {{η}^{->}}^{′} ) ] = {\sum }_{i} {[ {d}_{i} ( {t}^{->} , {η}^{->} ) - {d}_{i}^{( p )} ]}^{2} , \qquad Eq . ( 5 )$$

and components of the gradient of such an objective function are

$$\frac{\partial F}{\partial {t}_{j}} = 2 {\sum }_{i} ( {d}_{i} - {d}_{i}^{p} ) {\dot{D}}_{ij} {T}_{Δ x}^{{η}_{{k}_{ij}} / Δ x cos {θ}_{ij}}$$

$$and$$

$$\frac{\partial F}{\partial {η}_{k}} = 2 {\sum }_{i ∈ {I}_{k}} ( {d}_{i} - {d}_{i}^{p} ) {\sum }_{j} {\dot{D}}_{ij} {t}_{j} \frac{ln {T}_{Δ x}}{Δ x cos {θ}_{ij}} {T}_{Δ x}^{{η}_{{k}_{ij}} / Δ x cos {θ}_{ij}}$$

Determination of extrema of the quadratic objective function in Eq. (5) permits to demonstrate one example principle related to CBT: Dosage distribution delivered to a non-radially symmetric target (e.g., tumor) can be significantly improved with CBT. In one embodiment, a model of a brachytherapy source can be utilized and a lead radiation compensator with a thickness of less than 100 μm at most any location on the radiation compensator surface can be designed. In addition, in such embodiment, an example IMBT target can be an ellipsoidal tumor with an inferior-superior (I-S) length of about 10 cm, a right-left (R-L) width of about 6 cm, and a posterior-anterior (P-A) height of about 4 cm. In one aspect, the exemplary IMBT is designed to be of similar dimensions to the surface encompassed by a target region for brachytherapy of cervical cancer. A treatment plan for conventional BT can be generated and contrasted with a treatment plan for CBT generated in accordance with aspects described herein. In one aspect of an example implementation, such treatment plans can be generated by minimizing the quadratic objective function of Eq. (5) with definitions conveyed in accordance with Eq. (1), and under certain constraints, such as that the radiation compensator thickness does not exceed about 100 μm at most any location of the radiation concentrator surface and each dwell time of a plurality of dwell times for the radiation source (e.g., the model of the brachytherapy source) be greater than or equal to zero. In another aspect of the example implementation, the prescription dose can be configured to 100% for all voxels (or, more generally, finite regions) on the tumor surface. It is noted that in most computations (e.g., optimizations), voxels in the bulk of the tumor were excluded. The latter feature of implementation is typical in brachytherapy optimization or simulations in general, since position of the radiation source inside the tumor ensures that the dose inside the tumor is greater than the dose delivered at the surface.

FIG. 4 illustrates the resulting tumor surface dose distributions for radiation treatment of a tumor in accordance with one or more aspects. In one aspect, radiation is delivered to the surface of the posterior lobe of the tumor (e.g. an ellipsoidal tumor). The posterior-anterior, right-left, and inferior-superior ellipsoidal tumor dimensions are 4 cm, 6 cm, and 10 cm, respectively. In one treatment aspect, the tumor can be treated with a total of twenty-one dwell positions (e.g., j=1, 2, . . . , 18, 19, and 20) with 5 mm inferior-superior (I-S) spacing. An arrow oriented along the I-S direction indicates the direction of BT source displacement. Increasingly darker regions represent increasing magnitudes of underdose and overdose (see dosage scale labeled “Dose (%)” in FIG. 4), and white regions in the rendering receive the prescription dose (e.g., 100% value). The dosage scale (“Dose (%)”) is applicable to data in both charts 400 and 450. Chart 400 illustrates the resulting tumor surface dose distributions for conventional BT, e.g., without a radiation compensator of the disclosure. Chart 450 illustrates the resulting tumor surface dose distributions for CBT, e.g., in the presence of a radiation compensator in accordance with aspects of the subject disclosure. It is readily apparent from FIG. 4 that CBT can produce tumor surface doses that are substantively closer to the prescription (or prescribed dose) than conventional BT: In chart 450, the rendering of the tumor surface presents a larger area with white or light regions. In one aspect, the radiation compensator is a lead compensator having a thickness profile represented by the grayscale labeled “Compensator Thickness (μm)”. The thickness profile is conveyed in grayscale in the block labeled “Catheter and Compensator”.

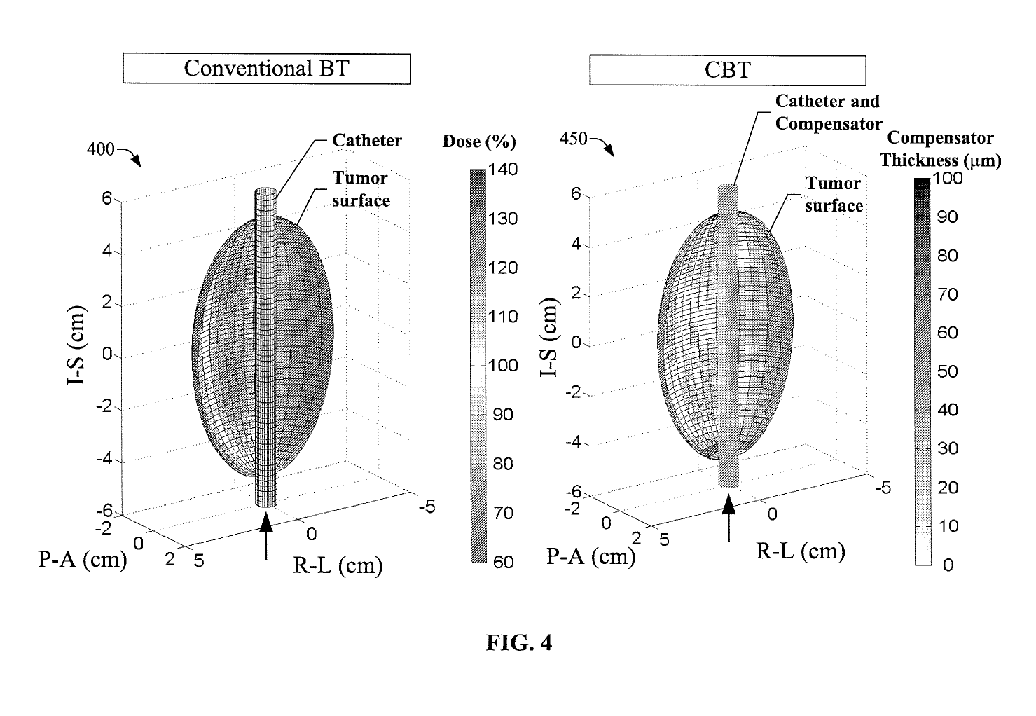

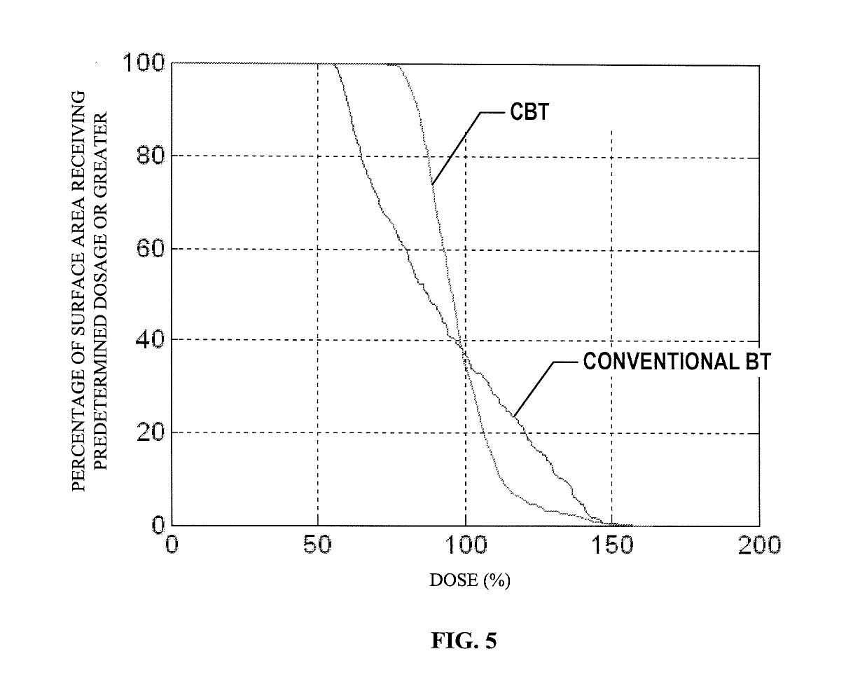

The dose-surface histograms in FIG. 5 demonstrate that, when both treatment methods (CBT and conventional BT) can deliver the prescription dose or greater to 40% of the tumor surface; 90% of the tumor surface receives doses of 60% and 80% of the prescription dose with conventional BT and CBT, respectively. Accordingly, utilization of CBT for treatment can provide about a 33% improvement over conventional BT in the illustrated exemplary implementation.

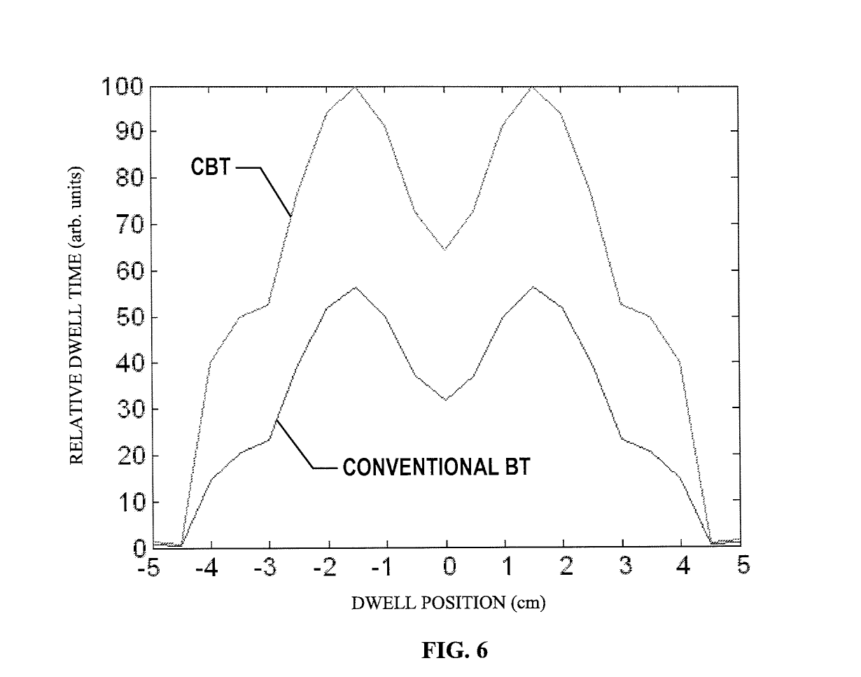

FIG. 6 illustrates computed (e.g., optimized) dwell times on a relative scale for the various source positions in an applicator, or CBT insertion device, for both conventional BT and CBT in accordance with one or more aspects described herein. The relative scale is normalized to the maximum dwell time in CBT. The total treatment time—which can be determined by the integral of a relative dwell time curve—for the ellipsoidal tumor case is about twice as long for CBT as it is for conventional BT. Without intending to be limited by theory, modeling, and/or simulation, it is believed that such difference originates in the radiation attenuation provided by the radiation compensator, which can prevent certain radiation source emissions from reaching the tumor.

FIGS. 7A-7B illustrates example thicknesses of a radiation compensator surface in accordance with one or more aspects described herein. In one aspect, the radiation compensator is fabricated from lead. The thicknesses are shown to-scale relative to the compensator spatial extent depicted in FIG. 7A, whereas a magnified rendering is presented in FIG. 7B. As illustrated, thickness are provided in units of μm.

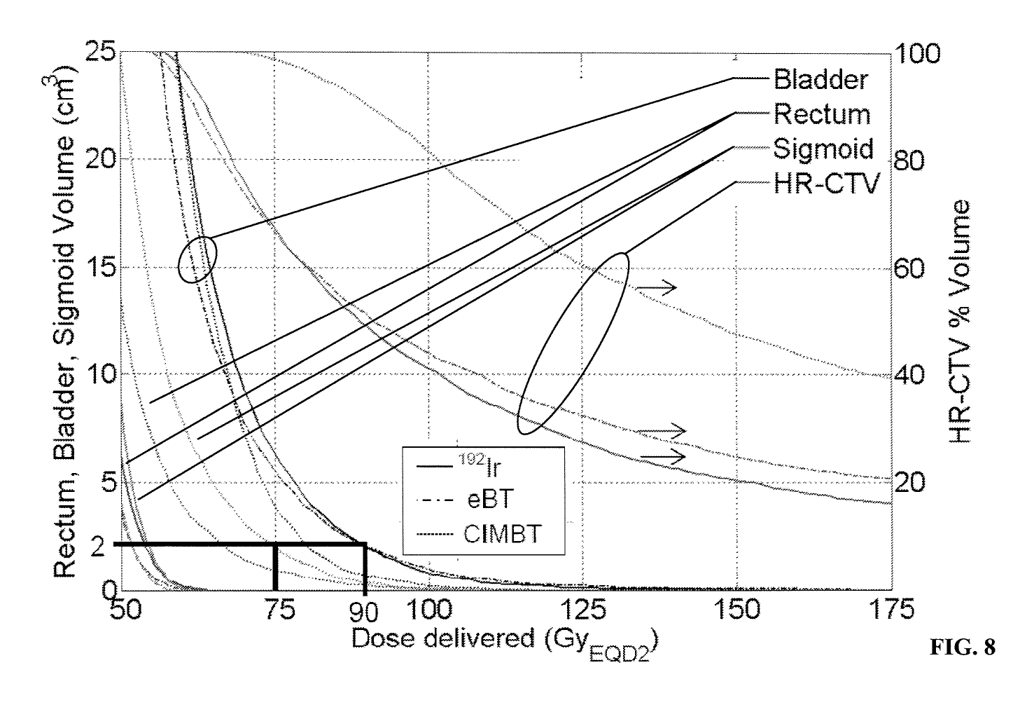

In other exemplary implementation, thicknesses at various locations of a radiation compensator surface can be determined for certain constraints related to dosage and organ anatomy. FIG. 8 illustrates dose-volume histograms for the organs depicted in FIG. 1B, such histograms determined in accordance with one or more aspects of the subject disclosure. HR-CTV doses are limited by the bladder dose constraint (e.g., about 90 Gy_{EQD2} to 2 cc) for ¹⁹²Ir-based BT and eBT, and the sigmoid dose constraint (e.g., about 90 Gy_{EQD2} to 2 cc) for the CBT case. In one aspect, D₉₀ for ¹⁹²Ir, eBT, and CBT are 64 Gy_{EQD2}, 62 Gy_{EQD2}, and 90 Gy_{EQD2}, respectively. Such constraints can be part of a radiation therapy plan.

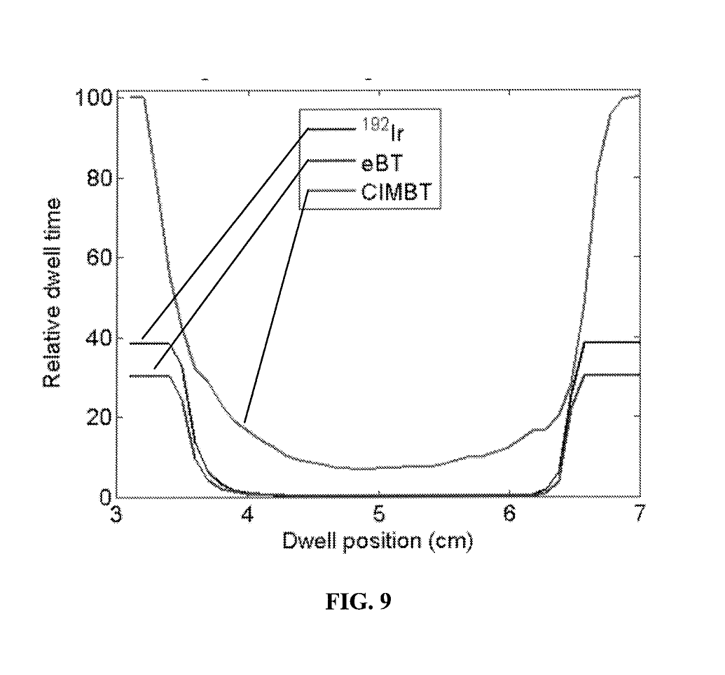

FIG. 9 illustrates relative dwell times for the three techniques for different radiation treatments—e.g., BT, eBT, and CBT—in accordance with one or more aspects of the disclosure. The relative scale being normalized to the maximum dwell time in CBT. In one aspect, dwell times at the HR-CTV ends are constrained by forcing the maximum divided by the mean dwell time to be 3 or less. It should be appreciated that such limitation is only exemplary and other conditions, or constraints, can be contemplated when determining (e.g., computing, optimizing, or the like) the dwell times in accordance with one or more aspects described herein. In one aspect, to determine the dwell times, it is assumed that the ¹⁹²Ir and eBT sources can have the same dosage rate in water at a distance of 4 cm lateral to the source axis. Therefore, the ¹⁹²Ir and eBT delivery times (or dwell times) can be comparable. In one aspect, the CBT dwell time can be greater than the delivery time for eBT by a factor of about 3.4. In certain embodiments, decreased CBT treatment times, e.g., integrated or accumulated dwell times, can be obtained at the cost of reducing D₉₀ in the HR-CTV. In one example practice scenario, a physician can select a D₉₀ in the range between the D₉₀ values for ¹⁹²Ir and CBT, wherein the D₉₀ can optimize a tradeoff between dwell time and HR-CTV conformity.

A thickness profile of a plurality of thicknesses for a respective plurality of locations in the surface of a radiation compensator also can be determined according to aspects described herein. FIG. 10 illustrates a thickness profile, which also can be referred to as a thickness distribution profile, of a tungsten attenuator assembled (e.g., mounted, coated, or otherwise integrated) on an optimized compensator used to generate the dose distribution depicted in FIG. 1C. Attenuator heights, or thicknesses, are shown on magnified scale with respect to size of the radiation compensator. The compensator can have a circular section and thus the circumferential position refers to a position on a segment defining the circumference of the circular section, whereas the longitudinal position refers to the position along an axis that pierces the circular section. In certain embodiments, the axis can be an axis of cylindrical symmetry of the compensator. As illustrated the largest thickness of the illustrated compensator is approximately 65 μm.

Various advantages emerge from the features or aspects of the disclosure convey that CBT of cervical cancer is feasible and can be beneficial in increasing delivery time of treatment and conformity of irradiation onto areas to be treated thus preserving surrounding healthy tissue. For example, the majority of patients having IB1-IV cervical cancerous tumors can be advantageously treated with the various embodiments of CBT described herein.

It should be appreciated that compensator-based intensity modulated brachytherapy can significantly improve cervical cancer dosage distributions without the need for supplementary interstitial BT. In one practice aspect, a physician can have freedom to optimize the tradeoff between increased delivery time and tumor dosage conformity with CBT. Since the high-Z (e.g., Z greater than or equal to 22) layers of compensators can be less than about 100 μm thick (see, e.g., FIG. 10), it can be expected that patient-specific compensators can be constructed rapidly (e.g., within a few minutes to less than one hour) in clinical situations, such during treatment, using, for example, circuit board printing technology, etching techniques, coating (e.g., evaporation, sputtering and sintering) milling, or the like), and so forth.

Various materials can be employed to produce a customized thickness profile of a radiation compensator described herein. The material can be a radiopaque material, which can comprise one or more of titanium, lead, gold, barium, barium sulphate, tungsten, bismuth, bismuth subcarbonate, tantalum, tin, iron, silver, molybdenum, platinum, and titanium. In other embodiments, the radiopaque material comprises one or more of a bismuth alloy, a tantalum alloy, a tin alloy, a silver allow, a molybdenum alloy, or a platinum alloy. In yet other embodiments, the radiopaque material comprises lead. In another embodiment, the radiopaque material further comprises one or more of lead powder or at least one etched lead sheets. In one embodiment, the radiopaque material comprises gold. In one aspect, the radiopaque material comprising gold can comprise gold nanoparticles. In another embodiment, the radiopaque material can comprise barium. In yet another embodiment, the radiopaque material comprises tungsten. In one aspect, tungsten can be present in the radiopaque material as tungsten powder. In certain embodiments, the radiopaque material comprises one or more of bismuth, tantalum, tin, silver, molybdenum, platinum, or titanium. In alternative or additional embodiments, the radiopaque material can comprise iron. In one aspect, iron can be present as iron powder or iron nanoparticles.

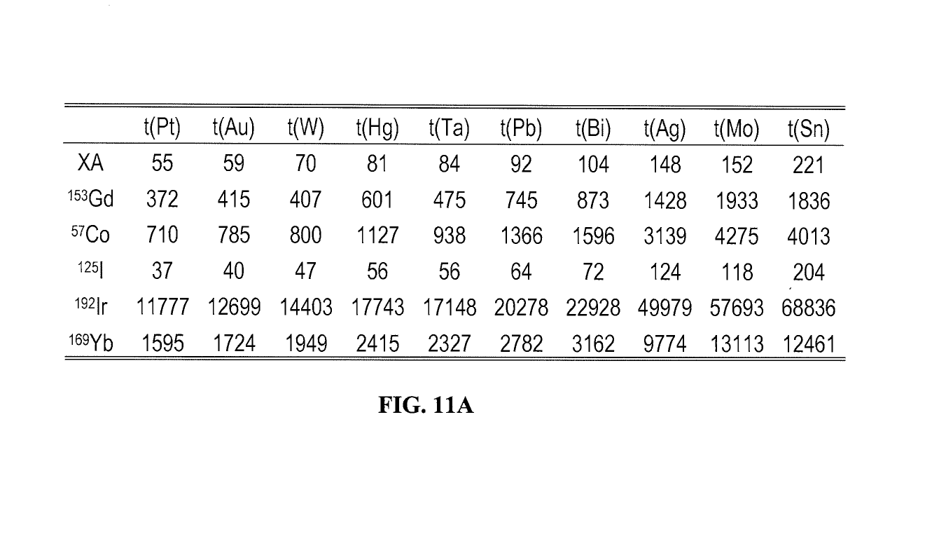

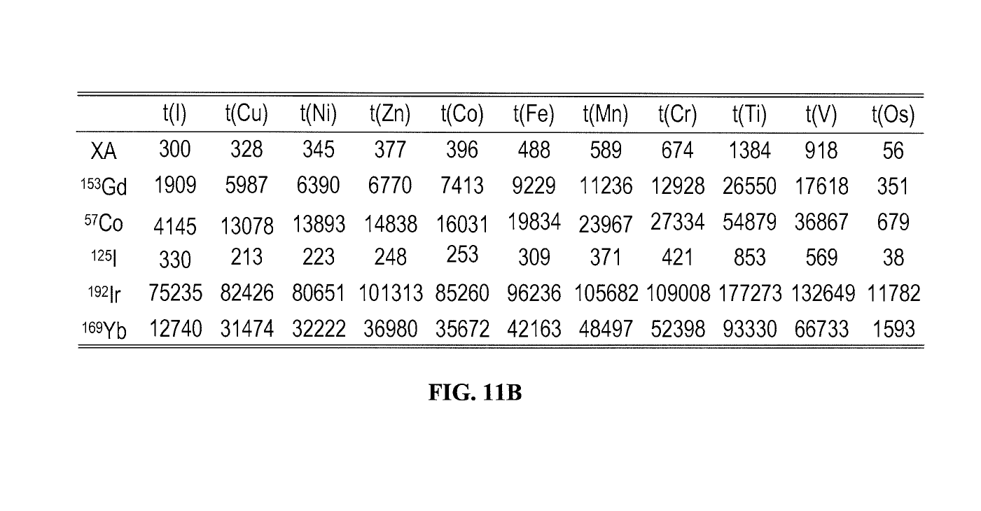

FIGS. 11A-11B illustrate example values of thicknesses of radiation compensator formed from different materials and for various BT sources in accordance with one or more aspects of the disclosure. Thicknesses for a material M (Pt, Au, W, Hg, Ta, Pb, Bi, Ag, Mo, Sn, I, Cu, Ni, Zn, Co, Fe, Mn, Cr, Ti, V, Os) are indicated as t(M) and presented in units of μm. Thicknesses for six sources are presented: Xoft Axxent (XA), ¹⁵³Gd, ⁵⁷ Co, ¹²⁵I, ¹⁹²Ir, and ¹⁶⁹Yb. In one aspect, thicknesses for the XA source are similar to thicknesses for a Zeiss IntraBeam® source. As described herein, the BT sources comprise an electronic source and radioisotopes. For a specific material, the thickness are presented in units of μm and permit energy transmission of nearly 10% when shielding respective BT sources. In one aspect, the thicknesses can be computed utilizing the definition of transmission factor for a specific compensator thickness presented in Eq. (2).

In addition, various equipment and systems can be exploited to fabricate a radiation compensator as described herein. As described herein, the attenuating material (e.g., radiopaque material, or semi-radiopaque material) can be printed or otherwise coated onto a surface of a radiation compensators that can be inserted into a delivery applicator of a device for radiation therapy. In certain embodiments, the attenuating material can be printing utilizing techniques similar, yet not identical to those employed for making printed circuit boards for computer components. In addition, since the thickness profile is customized to patient anatomy and to a region to be treated with radiation, such as a tumor, a thickness profile of a radiation compensator can break cylindrical or, more generally, radial symmetry of the radiation compensator and thus a mechanism or means for aligning the radiation compensator with a custom thickness profile as described herein can be needed prior to radiation delivery. In one aspect, such means for aligning the radiation compensator can include a small wire mounted on the inside of the applicator that, when aligned properly with the compensator, can send a signal to a user device or a control system (e.g., computer). In another aspect, the means for aligning can include a robust optimization algorithm that can produce compensators that mitigate or avoid sensitivity to misalignment.

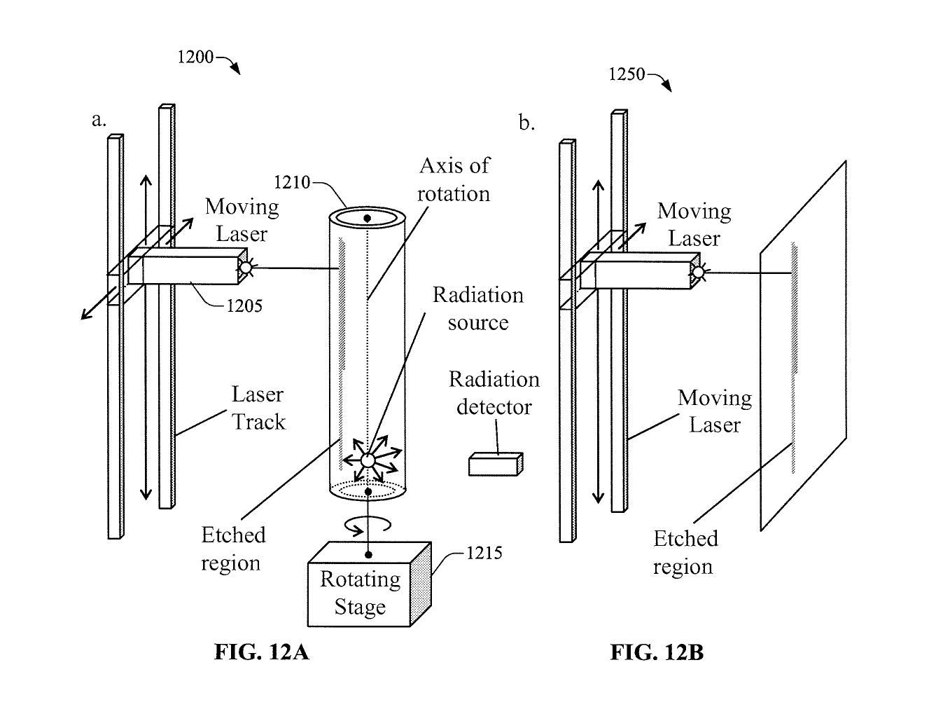

FIG. 12A illustrates an apparatus 1200 to fabricate a thickness profile of a radiation compensator in accordance with aspects described herein. In one aspect, the apparatus enables etching of the surface of a radiation compensator. In certain embodiments, such surface can be a non-treated surface—prior to etching—that can comprise a substrate of a radiopaque material, the radiopaque material can comprise a first high atomic-number material (e.g., Z greater than 21), a mixture of a plastic and a second high atomic-number material, a mixture of a rubber and a third high atomic-number material, or any combination thereof. A rotating stage unit 1215 (or rotating stage 1215) can comprise an electromechanical system configured to rotate the compensator 1210 and to control, at least, the operation of a laser 1205 that can etch the surface of the radiation compensator 1210. The laser 1205 can be configured to etched various portions of an exposed area of the radiation compensator. For example, the laser can be movably coupled or movably attached to a frame or a set of tracks that permits the laser to move in a plane. An automation system (which can be embodied in computer 2401; not shown in FIG. 12A) can control the operation of the laser and the rotating stage unit in order to achieve etching of a specific thickness profile (see, e.g., FIG. 7B) for a surface of the radiation compensator. In certain embodiments, the automation system can execute computer-executable instructions that cause a processor to energize the laser with a certain power, move the laser, and move (e.g., rotate) the radiation compensator. Such instructions can be programmed based on a desired thickness profile and through various programming techniques, which can be specific to the automation systems available to control the etching process.

In one aspect, exemplary apparatus 1200 can comprise a radiation source and a radiation detector system that can be included as part of a quality assurance stage being part of a manufacture of the radiation compensator. The quality assurance stage can comprise monitoring thickness of the etched region at one or more locations at such region. In one aspect, a radiation source can be inserted into the radiation compensator during the manufacturing process. The radiation source can be the same radiation source employed to implement a radiation treatment. Radiation emission from the radiation source and the radiation compensator can be detected outside of the radiation compensator and compared with expected measured values for radiation dose (see, e.g., FIG. 9, FIG. 6). In one aspect, if the measured radiation is lower than an expected or desired value of radiation, then the thickness of the radiation compensator is not adequate and adjustment to the etching or printing process can be effected. After adjustment of the etching or printing process, further radiation measurements can be conducted and as part of a feedback loop that ends after satisfactory measurements are accomplished. It should be appreciated that such quality assurance stage can be implemented in substantially any process for fabrication of a radiation compensator having a treated surface comprising a predetermined thickness profile.

Likewise, FIG. 12B illustrates an exemplary embodiment of an apparatus 1250 to fabricate a thickness profile of a radiation compensator in accordance with aspects described herein. Aspects of operation of apparatus 1250 are substantially the same as those of exemplary apparatus 1200. Yet, in exemplary apparatus 1250, etching of the surface of the radiation compensator is accomplished without utilization of the rotating stage. Instead, the surface of 1260 is etched in a planar arrangement. Such configuration is well suited for radiation compensators that can be manufactured from a flexible substrate, which can be etched prior to being bent into a specific geometry of the radiation compensator.

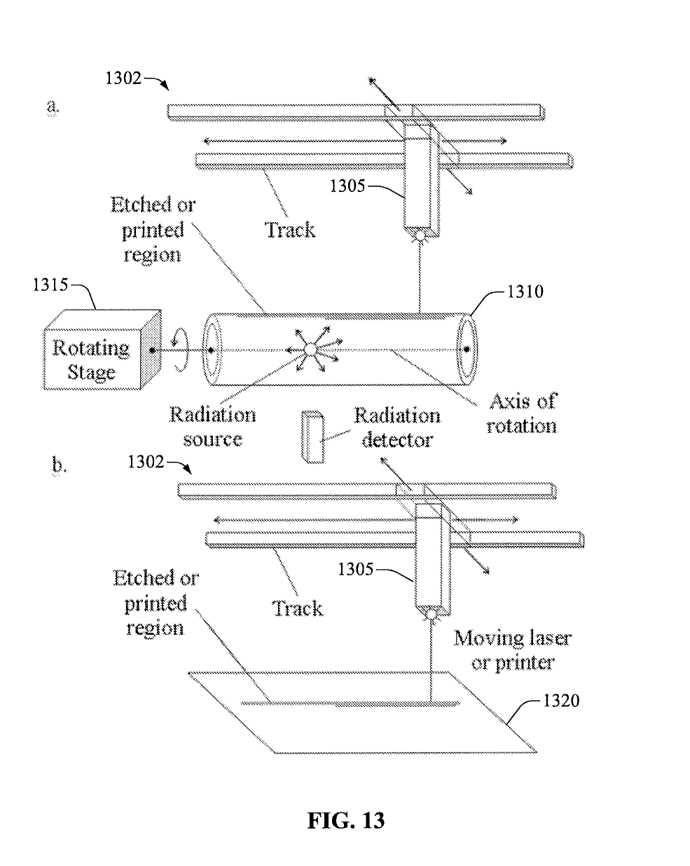

FIG. 13 illustrates exemplary embodiments of two example apparatuses that can etch or print a surface of a radiation compensator in accordance with aspects of the subject disclosure. In panel (a), the apparatus can enable etching, printing, or otherwise treating the surface of a cylindrical radiation compensator 1310. The apparatus can comprise a track 1302, means for treating the surface of the compensator 1310. For example, such means can comprise a laser and/or a printer 1305, and a rotating stage unit 1315 (or rotating stage 1315) which can comprise an electromechanical system configured to rotate the compensator 1310 and to control, at least, the operation of the means for treating the surface of the compensator. In panel (b), the apparatus can enable printing or etching a planar surface 1320 than can be folded into surface with a specific curvature suitable for forming the surface of a radiation compensator. In certain embodiments, the planar surface can be substrate of radiotransparent material. As described herein, the printing or etching of the planar surface 1320 are illustrative of various processes, such as milling, sintering (e.g., laser sintering described herein), sputtering, and the like, that can treat the planar surface 1320 to yield a treated surface having a predetermined (e.g., calculated as described herein) thickness profile in accordance with one or more aspect of the disclosure. In certain embodiments, the treated surface is a radiopaque material comprising at least one etched lead sheet.

In certain embodiments, an apparatus for providing a radiation compensator can comprise means for collecting data indicative of a position-dependent thickness profile; and means for providing a radiation compensator having a treated surface having a thickness according to the position-dependent thickness profile. Such profile can be determined in accordance with aspects of the disclosure. In one aspect, the means for providing the radiation compensator comprises means for etching a non-treated surface of the radiation compensator, wherein the non-treated surface is a substrate of a radiopaque material, the radiopaque material comprising at least one of a first high atomic-number material, a mixture of a plastic and a second high atomic-number material, and a mixture of a rubber and a third high atomic-number material. In another aspect, the means for etching comprises means for removing the radiopaque material in an amount effective to yield the thickness profile. In another aspect, the means for providing the radiation compensator comprises means for treating a non-treated surface of the radiation compensator with a radiopaque material, wherein the means for treating can yield the treated surface.

In another aspect, the non-treated surface of the radiation compensator 1310 or other non-treated surface can comprise a substrate of a radiotransparent material, and the means for treating comprises means for printing ink (e.g., laser or printer 1305) onto the substrate in an amount effective to produce the thickness profile, the ink containing the radiopaque material. In yet another aspect, the non-treated surface of the radiation compensator can comprise a substrate of a radiotransparent material, and wherein the means for treating comprises means for etching the substrate according to the thickness profile, wherein the means for etching yields an etched substrate.

In one aspect, the means for treating further comprises means for coating the etched substrate with a radiopaque material, and the means for treating further comprises means for sintering at least a portion of the radiopaque material.

In another aspect, the means for treating can comprise means for sputtering the non-treated surface of the radiation compensator with the radiopaque material, wherein the radiopaque material is a metal having a high atomic number (e.g., Z greater than or equal to 22). In certain embodiments, the radiopaque material comprises one or more of titanium, lead, gold, barium, barium sulphate, tungsten, bismuth, bismuth subcarbonate, tantalum, tin, iron, silver, molybdenum, platinum.

In other embodiments, the radiopaque material comprises lead. In another embodiment, the radiopaque material further comprises one or more of lead powder or at least one etched lead sheets. In one embodiment, the radiopaque material comprises gold. In one aspect, the radiopaque material comprising gold can comprise gold nanoparticles.

In another embodiment, the radiopaque material can comprise barium. In yet another embodiment, the radiopaque material comprises tungsten. In one aspect, tungsten can be present in the radiopaque material as tungsten powder.

In certain embodiments, the radiopaque material comprises one or more of bismuth, tantalum, tin, silver, molybdenum, or platinum. In alternative or additional embodiments, the radiopaque material can comprise iron. In one aspect, iron can be present as iron powder or iron nanoparticles.

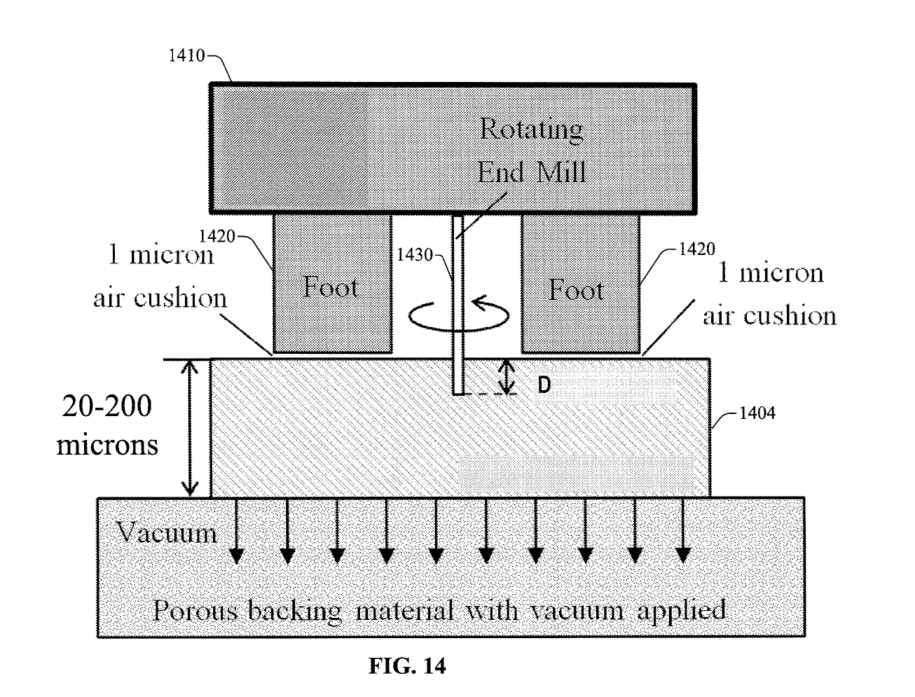

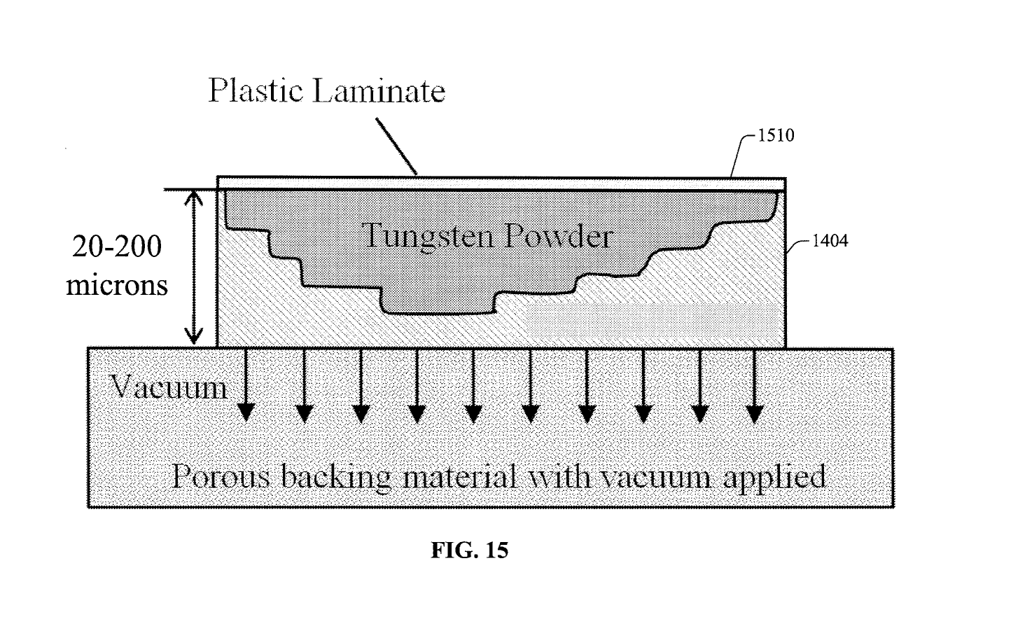

FIG. 14 illustrates an example embodiment of an assembly 1400 to produce a compensator for CBT in accordance with aspects described herein. In one aspect, the assembly can produce the compensator by milling one or more pockets out of slab 1404 of solid material, such a plastic, utilizing a circuit board plotter 1410 (such as a ProtoMat S103 circuit hoard plotter from LPKF of Garbsen, Germany). In one aspect, such slab 1404 can be a thin film having a thickness similar to the largest thickness, e.g., about 60 μm to about 200 μm (see, also FIGS. 12A-12B), of a thickness profile intended to be produced on the surface of a radiation compensator. In addition, at least one of the one or more pockets can be filled with a radiopaque material, such as small-grain (e.g., from about 1 μm to about 1.5 μm) tungsten powder. As illustrated in FIG. 15, the slab with filled pocket(s) can be laminated, with a plastic laminate 1510, to provide a thin plastic adhesive film forming a laminated compensator for CBT. In certain implementations, the one or more pockets are intended to have a thickness accuracy for the radiopaque material of ±3 μm at most positions on the laminated compensator. Such thickness accuracy can be measured by imaging the unwrapped compensator with digital fluororadiography.

In scenarios in which the circuit board plotter can mill pockets into the slab of solid material (e.g., plastic sheets) with a depth accuracy of approximately 10% or better of a maximum thickness (see, e.g., FIGS. 12A-12B) in a desired thickness profile, it may be feasible to fabricate radiation compensators in accordance with one or more aspects of the disclosure. In addition or in the alternative, in scenarios in which the rotating end mill 1430 in the assembly can generate pockets in the slab 1404 (e.g., a plastic sheet) at accuracies in the horizontal plane of about 10% or better of the maximum thickness in the desired thickness profile, it may be feasible to produce radiation compensators in accordance with one or more aspects of the disclosure. As illustrated, the rotating end mill 1430 can penetrate up to about a distance D from atop a surface of the slab 1404. Such accuracy in the horizontal plane can be satisfactory, in certain implementations, for CBT devices having a footprint of each compensator element of the order of 1 mm×1 mm. In embodiments in which the mill depths can be determined relative to the location of a mechanical foot 1420 that can be placed on, for example, an air cushion (as depicted in FIG. 14), the pocket depths can be defined relative to the surface of the slab of solid material (e.g., a plastic sheet). In such embodiments, it may be feasible to fabricate radiation compensators in accordance with one or more aspects of the disclosure.

It should be appreciated that the milling process described herein is one example of various processes (e.g., sputtering) that can treat a non-treated surface, which can be an initial surface of a radiation compensator, to produce a specific thickness profile of a radiopaque material. In certain embodiments, instead of milling a slab of a solid material (e.g., a plastic or an intrinsic semiconductor), such slab can be etched to remove material from the slab and form an etched slab having a predetermined depth profile. Such depth profile can be complementary representation of an intended thickness profile. Accordingly, the etched slab can be coated (e.g., via sputtering or other deposition process) with a radiopaque material to form a predetermined thickness profile that can shield radiation and permit CBT according to one or more aspects described herein.

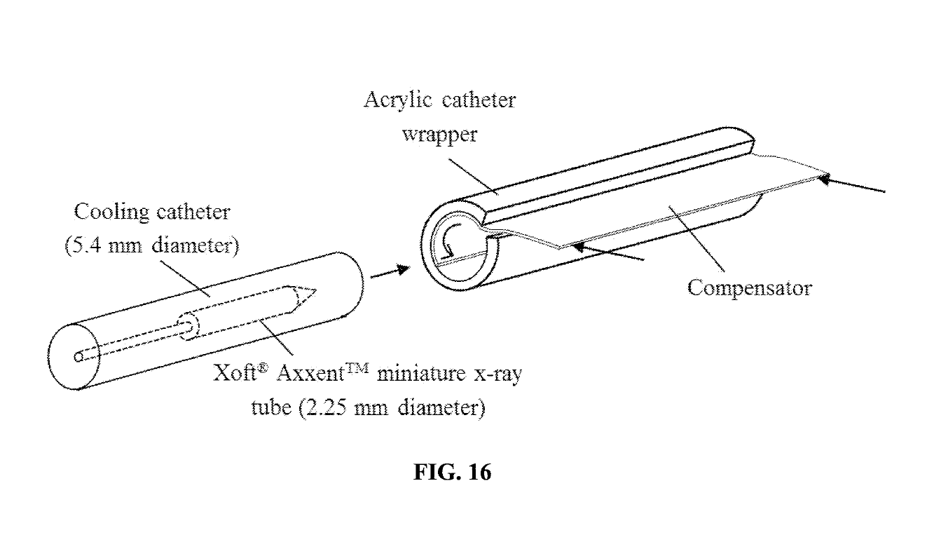

A portion of a compensator that can be produced through the assembly depicted in FIG. 14 is illustrated in FIG. 15. While the portion of the compensator is illustrated with tungsten powder in FIG. 15, other radiopaque materials, such those indicated in FIGS. 12A-12B, alloys thereof, and other metals having atomic number greater than or equal to 22—can be utilized to fill milled pockets an thus produce a radiopaque layer with a specific thickness profile as described herein. In addition or in the alternative, agglomerates of nanoparticles formed from a radiopaque material can be utilized to fill a milled pocket. The compensator can be assembled (e.g., wrapped) around a radiation source by utilized various means for assembling the compensator. Such means can include a device according to the diagram illustrated in FIG. 16.

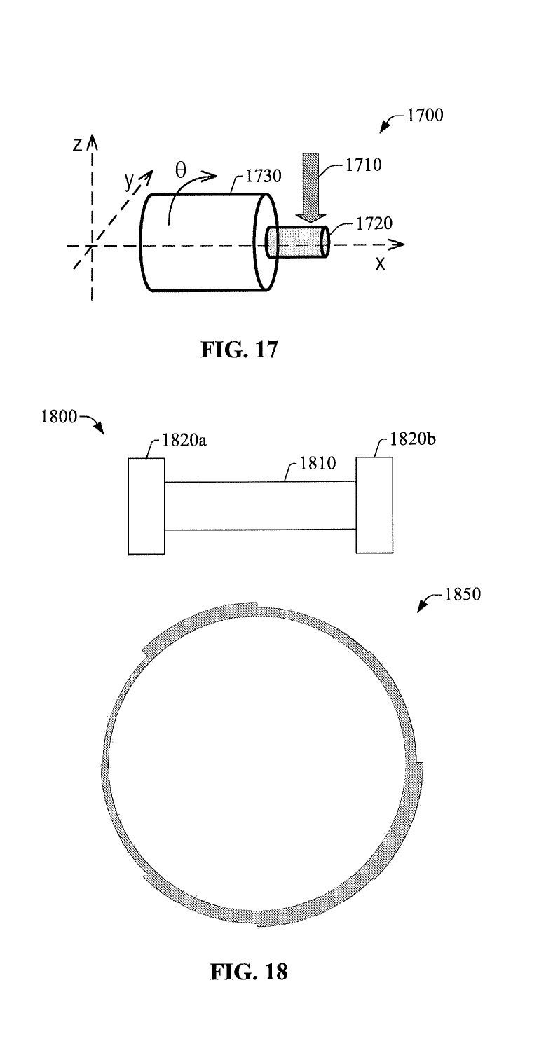

In additional or alternative embodiments, a milling process can be utilized to treat the surface of a radiopaque material and, in response to treatment, yield a radiation compensator having a thickness distribution based at least on a specific area to be irradiated and specific radiation therapy. FIG. 17 depicts an example embodiment of a milling apparatus 1700 that can permit fabrication of a radiation compensator in accordance with one or more aspects of the disclosure.

In the illustrated embodiment, the milling apparatus 1700 comprises a milling member 1710 that performs the milling and can move along a first direction (e.g., z axis) normal to the surface of a radiopaque material to be milled to form the radiation compensator. It should be appreciated that the milling member 1710 can rotate about the direction normal to the surface of such material. In addition, the milling apparatus 1700 comprises a stock member 1730 that can hold the radiopaque material. In one aspect, the stock member 1730 can rotate an angle θ about a second direction (e.g., x axis) and translate along one or more of the first direction, the second direction, or a third direction (e.g., y axis). Such translational and rotation degrees of freedom can permit the milling member 1710 to remove material from the radiopaque material 1720 on substantially any position on the surface of the radiopaque material 1720. In should be appreciated that the milling apparatus 1730 has four degrees of freedom and thus it is referred to as “4D milling” apparatus. In one aspect of the illustrated embodiment, the milling apparatus 1700 can remove material with a depth accuracy of approximately 2.5 μm, which can provide a resolution suitable for generation of a thickness distribution as described herein (see, e.g., FIG. 11).

Operation of the milling apparatus 1700 can be automated in order to fabricate the radiation compensator according to a predetermined specification—e.g., a compensator suitable for treatment of a specific area with a specific radiation treatment). In certain implementations, automation can comprise generation of a design of a thickness profile to be milled onto the surface of the radiopaque material 1720. For example, the design can be produced with a suitable industrial design generation software application. As part of the automation, the design can converted to a suitable set of one or more computer-executable instructions (e.g., programming code instructions) that can be executed by a computing device (e.g., a controller) that is functionally coupled to the milling apparatus 1700 and, in response to execution, the computing device can control the milling apparatus 1700 to fabricate a radiation compensator according to the design. In one aspect, prior to automated milling, the stock member 1730 coupled to the radiopaque material 1720 can be suitable positioned (e.g., centered and the coordinates of the apparatus calibrated).

In certain implementations, the radiopaque material can be a titanium rod and the designed radiation compensator can have two end caps. In one aspect, the end caps can permit the radiopaque material to be mounted or otherwise fitted to the stock member 1730 via an adapter sleeve in such member in order to mill a predetermined thickness profile. In one aspect, the titanium rod can have a 0.5 in. diameter.

Diagram 1800 in FIG. 18 illustrates a side view of the radiopaque material 1720 (e.g., the titanium rod) in accordance with one or more aspects of the disclosure. Each end cap 1820a and 1820b comprises a 0.2 in. long tube having an inner diameter (ID) of 0.22 in. and an outer diameter (OD) of 23/64 in. The main section of the radiopaque material can form the main section 1810 of the radiation compensator resulting from milling. As part of the milling, in one aspect, the radiopaque material can be milled to form a tube (e.g., the tube having ID equal to 0.22 in. and an OD equal to 23/64 in., and a length of 2.4 in.) by using a conventional milling machine. The main section can attenuate radiation and can lie, in one aspect, concentrically between the two end caps. In another aspect, the main section can comprise a 2 in. long tube with ID of 0.22 in. and varying OD according to a milled thickness profile. Diagram 1850 illustrates a cross-sectional view of the radiation compensator main section. As illustrated, the radiation compensator has a “pie-shaped” thickness profile. It should be appreciated that the foregoing dimensions are illustrated and are not intended to be limiting of radiation compensators that can be fabricated through milling.

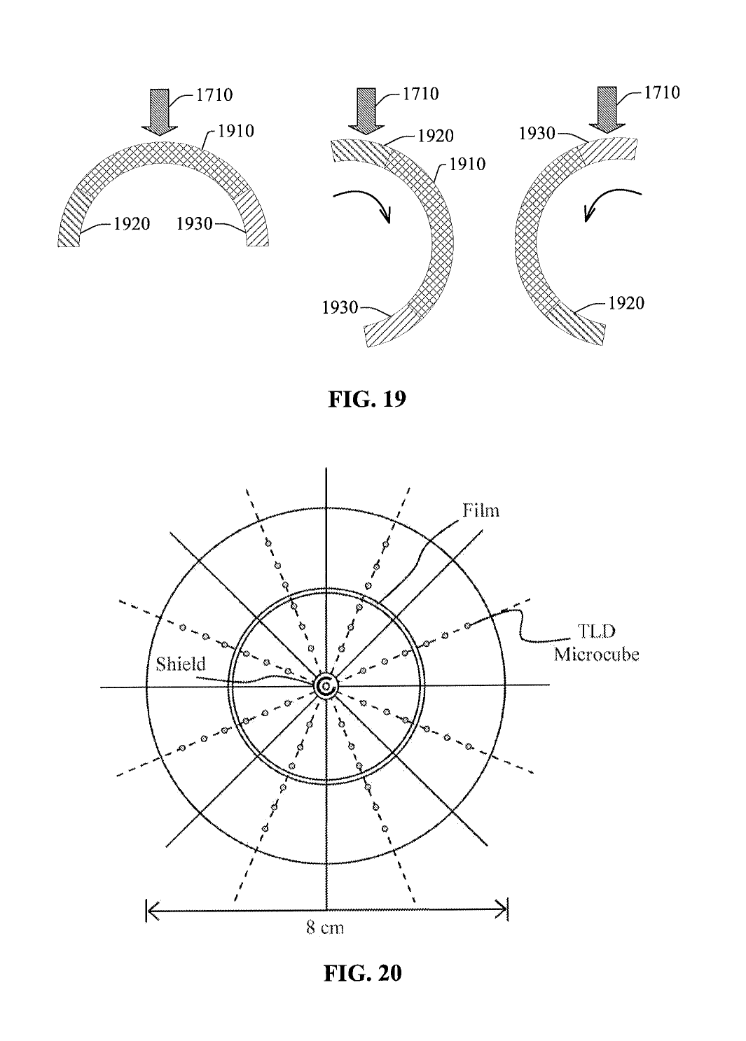

In 4D milling, milling time can be a factor affecting performance of fabrication of a radiation compensator. In an idealized scenario, a divergently large number of cuts performed with the milling member 1710 can be necessary to remove material from the radiopaque material 1720 and obtain a predetermined thickness profile of a radiation compensator (e.g., compensator main section 1810). Such large number of cuts, however, can incur a substantial milling time interval (e.g., hours). Thus, in one implementation scenario, number of cuts performed with the milling member 1710 can be reduced with the ensuing reduction of incurred milling time. For example, for milling each “pie section” of the example radiation compensator illustrated in diagram 1850, an 1/16 in. diameter mill member embodying the mill member 1710 can mill out a portion of radiopaque material to an intended depth in a first section of the radiopaque material 1720 (e.g., a middle section 1910), then the stock member 1730 can rotate clockwise (indicated with an arrow in FIG. 19) to permit the mill member 1710 to cut a second section (e.g., a left section 1920) and, after such cut, the stock member 1730 can rotate counterclockwise (indicated with another arrow) to permit the mill member 1710 to cut a third section (e.g., the right section 1930). In such implementation, the mill member 1710 can effect three cuts for each “pie section”, which can substantially shorten the milling time for each “pie section” from about 30 minutes to about 50 minutes.

As described herein, the thickness profile of a compensator for CBT can be determined based on data indicative of a specific radiation treatment in order to attain a predetermined radiation dosage at a tumor or tissue to be treated. Accurate dose calculation software (e.g., compensator design software 1706) or firmware can be exploited to predict dose distributions within a specific accuracy (e.g., 5% deviation) in order to provide radiation treatment safely. In one aspect, to monitor radiation dosage for a specific compensator, dose distribution produced by the compensator can be measured in a phantom, referred to as a quality assurance (QA) phantom. In one embodiment, an acrylic QA phantom can be utilized in such measurements. In one aspect, the acrylic phantom can comprise two 4 cm thick acrylic cylindrical inserts having a cross section as illustrated in FIG. 20. In another aspect, measurement of dose distribution in such phantom can be performed by utilizing a Gafchromic EBT2 film that is inserted into a circular film slot of the phantom. In aspect, the phantom contains eight spokes with pits, separated from each other by about 5 mm, containing 1×1×1 mm³ thermoluminescence dosimeter (MD) microcubes that pass through the central axis. The microcubes can be utilized to convert the film measurements to units of radiation dose with an accuracy of about ±5%.

Other assemblies can be utilized to produce compensators for CBT. For example, an apparatus for laser sintering can be utilized to treat a surface of a radiopaque material. Laser sintering can be implemented as an additive metal fabrication technology. In one aspect, laser sintering can produce a plurality of layers by laser-sintering very fine layers of metal powders on a layer-by-layer basis, permitting a gradual build-up of a solid structure (e.g., a metallic structure) according to a predetermined thickness profile as described herein. In one implementation of a laser sintering cycle, an initial layer of fine metal powder can be deposited onto a platform inside the apparatus for laser sintering. The initial layer can be sintered using a laser, such as a diode pump fibre optic laser, that can be controlled in a plane parallel to the platform in order to achieve a predetermined part shape and associated feature tolerances. An additional layer of metal powder can be deposited on top of the sintered initial layer, can be sintered by the laser to form a bond with the initial layer. The process can continue with deposition of a further layer of metal powder onto a previously sintered layer and sintering of the newly deposited layer. Other layers can be deposited sintered to a group of previously sintered layers.

To fabricate a compensator via laser sintering, in one aspect, a design of a desired radiation compensator can be supplied to a computing device (e.g., a controller) functionally coupled to or included in an apparatus for laser sintering. Based on the design, the computing device can generate a set of computer-executable instructions that, in response to execution (e.g., by the controller), can cause the apparatus for laser sintering to generate and sinter a plurality of layers having thicknesses according to the design. In one embodiment, the desired radiation compensator can be fabricated by laser sintering layers formed from cobalt-chrome powder. In another embodiment, the desired compensator can be fabricated by laser sintering layers formed from titanium powder. In the Ti-based embodiment, thickness resolution ranges from about 0.002 in. to about 0.005 in.

The various aspects of the subject disclosure provide a device for CBT. In certain embodiments, such device comprises a radiation compensator having a treated surface having a position-dependent thickness according to a thickness profile based on a radiation therapy plan and geometry of a region to be treated; and a source of radiation movably inserted into a first enclosure coupled to the radiation compensator, wherein the radiation source is adapted to reside at a plurality of locations within the radiation compensator during a respective plurality of periods, each period of the plurality of periods being equal to a respective dwell time of the plurality of dwell times, and wherein each dwell time is based on the radiation therapy plan.

In certain embodiments, the radiation compensator resides within a second enclosure that encompasses the first enclosure, the first enclosure adapted to move relative to the second enclosure, and wherein the second enclosure is coupled to alignment means (e.g., key or indexing unit 2110 and guide 2120) for positioning the first enclosure relative to the second enclosure. It should be appreciated that the thickness profile of a surface of the radiation compensator can break cylindrical symmetry thereof as a result of the thickness profile being tailor to a patient's anatomy and, more specifically, to an area of tissue affected by a tumor. Accordingly, orientation or alignment of the radiation compensator is important for adequate radiation therapy.

The alignment means can be manufactured of any material that can imaged with one or more 3D imaging techniques, such as one or more of USI, MRI, CT, PET, or the like. In one aspect, as illustrated in FIG. 21, the alignment means for positioning the first enclosure relative to the second enclosure comprises: means for indicating orientation of the second enclosure relative to the region to be treated; and means for locking at least part of the first enclosure outside the second enclosure in response to misalignment between orientation of the first enclosure and the orientation of the second enclosure. In another aspect, the means for indicating orientation of the second enclosure relative to the region to be treated are adapted to be visible on an three-dimensional imaging system. In another aspect, the first enclosure is a catheter (see, e.g., FIG. 21), the source of radiation is movably inserted into the catheter via insertion means, and the second enclosure is an applicator composed of a flexible biocompatible material. In certain embodiments, the device can include a radiation compensator that resides outside the catheter. In one aspect, the radiation compensator resides within the catheter.

As described supra, the radiation compensator attenuates radiation emanating from a radiation source. To at least such end, the radiation compensator in the device of the subject disclosure can be coated with a radiopaque material. In one aspect, the radiopaque material is a metal having a high atomic number (e.g., atomic number greater than or equal to 22). In another aspect, the radiopaque material is one of lead, gold, barium, barium sulphate, tungsten, bismuth, bismuth subcarbonate, tantalum, tin, iron, silver, molybdenum, platinum. In certain embodiments, the radiopaque material is a combination of such materials. Radiopaque materials utilized in the radiation compensator can be polycrystalline or monocrystalline. In addition, such materials can include nanoparticles or particulate matter of various sizes (e.g., particles with sizes of the order of a few to several microns).

When delivering CBT for treating a disease such as cervical cancer, a brachytherapy applicator, through which the radiation source travels, in one embodiment, is inserted into the patient prior to an image acquisition step, which is critical for treatment planning. The imaging system could be computed tomography, magnetic resonance imaging, or ultrasound, for example. In one embodiment, it is important that the applicator is in place during the imaging process, since the applicator is what geometrically constrains the radiation-emitting brachytherapy source during the treatment process. Without detailed imaging information on the applicator location relative to the cancer under treatment, and sensitive normal tissues such as rectum, bladder, and sigmoid colon, it is not possible, in one embodiment, to determine either how the compensator should be shaped or how long the source should stop at each planned position inside the applicator.

Once a patient-specific and treatment-specific compensator has been fabricated, a challenge associated with CBT delivery is inserting a patient-specific compensator into the applicator, which is often curved to match the patient's anatomy. A system for CBT delivery that enables compensator placement inside of a curved applicator is described below.

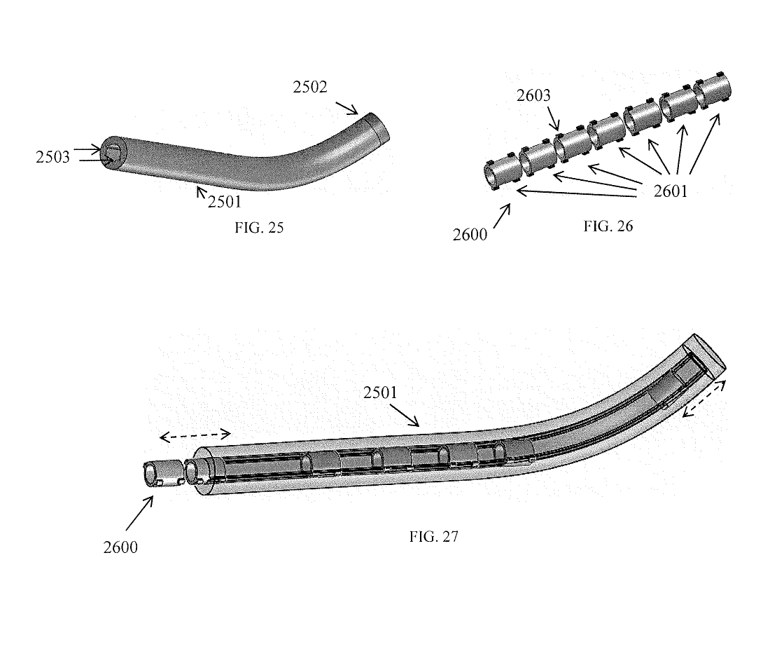

The curved CBT applicator system may comprise, in one embodiment, (1) a CBT applicator 2501 that includes a removable cap 2502 at the end (FIG. 25), and (2) a compensator 2600 with multiple segments 2601 (FIG. 26). In one embodiment, two notches 2503 are present lengthwise along the applicator 2501 inner surface. Protrusions 2603, which are relatively short compared to the length of each compensator segment 2601, are constructed on the outer surface of the compensator 2600 using the same technique as the rest of the compensator 2600; for example, with direct metal laser sintering (DMLS). The notches 2503 and protrusions 2603 slide along a track comprising a lock system that ensures the compensator 2600 will stay at a fixed orientation inside the applicator 2501 (FIG. 27).

In another embodiment, more than two notches 2503 are present along the inner surface of the applicator 2501, enabling an angularly-alternating pattern of notches 2503 on the outer compensator 2600 on the plane perpendicular to applicator 2501 axis. Such an approach distributes the protrusions 2603 in a manner that reduces the impact of the attenuation due to the protrusions 2603 on the radiation dose distribution in the patient. As the dosimetric effect of the protrusions 2603 can be accounted for in the CBT treatment planning process, the compensator 2600 thicknesses in the non-protrusion regions can be designed to offset the dosimetric impact of the protrusions 2603.

The CBT delivery process may, in one embodiment, entail inserting the individual compensator 2600 segments into the applicator 2501 and using a flexible plastic tube to push them to the distal end of the applicator 2501. After the treatment is finished, the applicator 2501 may be removed from the patient, the end cap 2502 may be unscrewed from the applicator 2501, and the compensator 2600 segments are pushed out of the applicator 2501 using a flexible plastic rod.

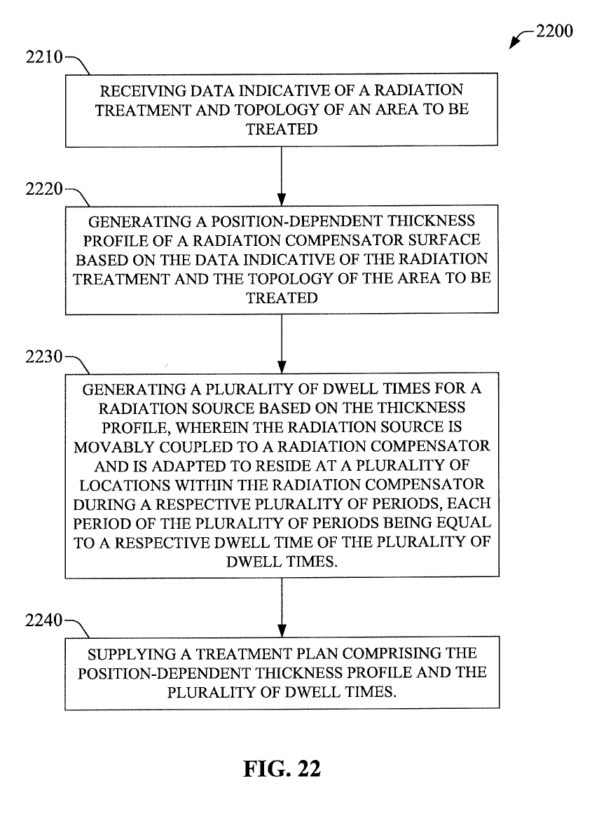

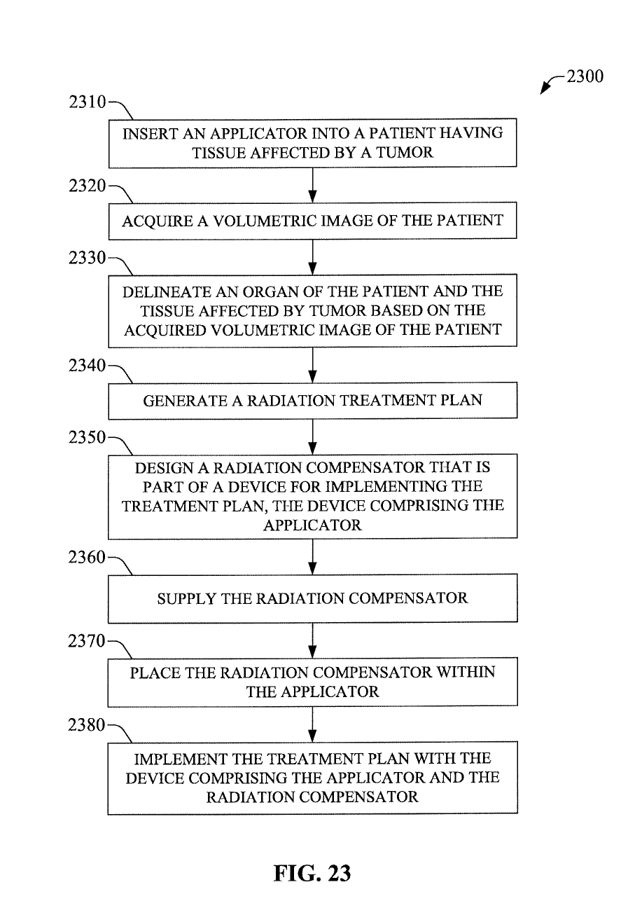

In view of the aspects described hereinbefore, an exemplary method that can be implemented in accordance with the disclosed subject matter can be better appreciated with reference to the flowchart in FIGS. 22-23. For purposes of simplicity of explanation, the exemplary method disclosed herein is presented and described as a series of acts; however, it is to be understood and appreciated that the claimed subject matter is not limited by the order of acts, as some acts may occur in different orders and/or concurrently with other acts from that shown and described herein. For example, the various methods or processes of the subject disclosure can alternatively be represented as a series of interrelated states or events, such as in a state diagram. Moreover, when disparate functional elements implement disparate portions of the methods or processes in the subject disclosure, an interaction diagram or a call flow can represent such methods or processes. Furthermore, not all illustrated acts may be required to implement a method in accordance with the subject disclosure. Further yet, two or more of the disclosed methods or processes can be implemented in combination with each other, to accomplish one or more features or advantages herein described. It should be further appreciated that the exemplary methods disclosed throughout the subject specification can be stored on an article of manufacture, or computer-readable medium, to facilitate transporting and transferring such methods to computers for execution, and thus implementation, by a processor or for storage in a memory.

FIG. 22 is a flowchart of an exemplary method 2200 for providing a radiation compensator in accordance with aspects of the subject disclosure. A computer or computing device can implement (e.g., execute) exemplary method 2200. In one aspect, a processor within or functionally coupled to the computer or computing device can be configured to execute computer-executable instructions and, in response to execution, the processor can carry out the various steps that comprise exemplary method 2200. Similarly, yet not identically, the computer or computing device can execute the various methods, or portion(s) thereof, disclosed herein. Exemplary method can comprise various steps. At step 2210, receiving data indicative of a radiation treatment and topology of a region to be treated. At step 2220, generating a position-dependent thickness profile of a radiation compensator surface based on the data indicative of the radiation treatment and the topology of the region to be treated. In one aspect, step 2220 can comprise discretizing the radiation compensator surface into a plurality of voxels and assigning a respective initial plurality of thicknesses to the plurality of voxels. In another aspect, step 220 also can comprise determining an extremum of an objective function (such as F[\vec{d}(\vec{t}′, \vec{η}′)] in Eq. (5)) by iteratively updating each thickness of the respective initial plurality of thicknesses and each dwell time of an initial plurality of dwell times, wherein the objective function is indicative of a difference among a prescribed dose at a position in the region to be treated and an actual dose provided at the position, the updating step yielding a current plurality of thicknesses and a current plurality of dwell times. In one aspect, in response to identifying the extremum, exemplary method 2200 can comprise performing the steps of configuring the current plurality of thicknesses as the position-dependent thickness profile (see, e.g., FIG. 10); and configuring the current plurality of dwell times as the plurality of dwell times.

At step 2230, generating a plurality of dwell times for a radiation source based on the thickness profile, wherein the radiation source is movably coupled to a radiation compensator and is adapted to reside at a plurality of locations within the radiation compensator during a respective plurality of periods, each period of the plurality of periods being equal to a respective dwell time of the plurality of dwell times. At step 2240, supplying a treatment plan comprising the position-dependent thickness profile and the plurality of dwell times.

In certain embodiments, exemplary method 2200 can further comprise providing a radiation compensator having a treated surface having a thickness according to the position-dependent thickness profile, wherein providing the radiation compensator comprises etching a non-treated surface of the radiation compensator, wherein the non-treated surface is a substrate of a radiopaque material, the radiopaque material comprising at least one of a first high atomic-number material, a mixture of a plastic and a second high atomic-number material, and a mixture of a rubber and a third high atomic-number material.

In one aspect, the etching step comprises removing the radiopaque material in an amount effective to yield the thickness profile, wherein providing the radiation compensator comprises treating a non-treated surface of the radiation compensator with a radiopaque material, wherein the treating step yields the treated surface.

In certain embodiments, in addition to providing the radiation compensator, exemplary method 2200 can further comprise aligning the radiation compensator inside an applicator configured to implement at least part of the radiation treatment. In other embodiments, exemplary method 2200 can further comprise monitoring thickness of the treated surface in response to the treating step and at one or more locations in the treated surface. The monitoring step can be implemented by an automation control system (e.g., a Programmable Logic Controller with suitable logic or, more generally a computing device such as computer 2401 programmed with suitable logic retained in system memory 2412) that controls an X-ray diffraction system or other equipment suitable for measuring thickness of the treated surface. In one aspect, the non-treated surface of the radiation compensator comprises a substrate of a radiotransparent material, and wherein the treating step comprises printing ink onto the substrate in an amount effective to produce the thickness profile, the ink containing the radiopaque material. In another aspect, the treating step can comprise painting a high-density material onto the substrate in an amount effective to produce the thickness profile, wherein the high-density material can contain the radiopaque material or can be an opaque or semi-opaque to radiation. In another aspect, wherein the non-treated surface of the radiation compensator comprises a substrate of a radiotransparent material, and wherein the treating step comprises etching the substrate according to the thickness profile, wherein the etching step yields an etched substrate. In the various embodiments of exemplary method 2200, the radiopaque material can be one of the various materials described herein or any combination thereof.

In certain embodiments, the treating step further comprises coating the etched substrate with a radiopaque material, wherein the treating step further comprises sintering at least a portion of the radiopaque material. In the alternative or in addition, the treating step can comprise sputtering the non-treated surface of the radiation compensator with the radiopaque material.

FIG. 23 is a flowchart of an exemplary method 2300 for conducting therapeutic treatment with a medical device (also referred to as a device) for implementing radiation therapy in accordance with aspects described herein. In one aspect, the medical device is a brachytherapy device. Yet, other medical devices for implementing radiation therapy also are contemplated. At step 2310, an applicator is inserted into a patient having tissue affected by a tumor. At step 2320, a volumetric image of the patient is acquired. The volumetric image can be a three-dimensional image obtained through at least one MRI, CT, PET, ultrasound echography or imaging, or the like. At step 2330, an organ of the patient and the tissue affected by the tumor is delineated based on the acquired volumetric image of the patient. At 2340, a radiation treatment plan is generated. Such plan can be generated in accordance with various aspects described herein. At act 2350, a radiation compensator that is part of the medical device for implementing the treatment plan is designed, the medical device comprising the applicator. At act 2360, the radiation compensator is supplied. The radiation compensator has one or more features described herein and can be supplied in accordance with various aspects of the subject disclosure; for instance, various aspects of exemplary method 2300 can enable supplying the radiation compensator. At act 2370, the radiation compensator is placed within the applicator (or, more generally, an insertion device). Placing the radiation compensator can be accomplished with one or more insertion means, such as a wire or a movable shaft. At act 2380, the treatment plan is implemented with the device comprising the applicator or the radiation compensator.

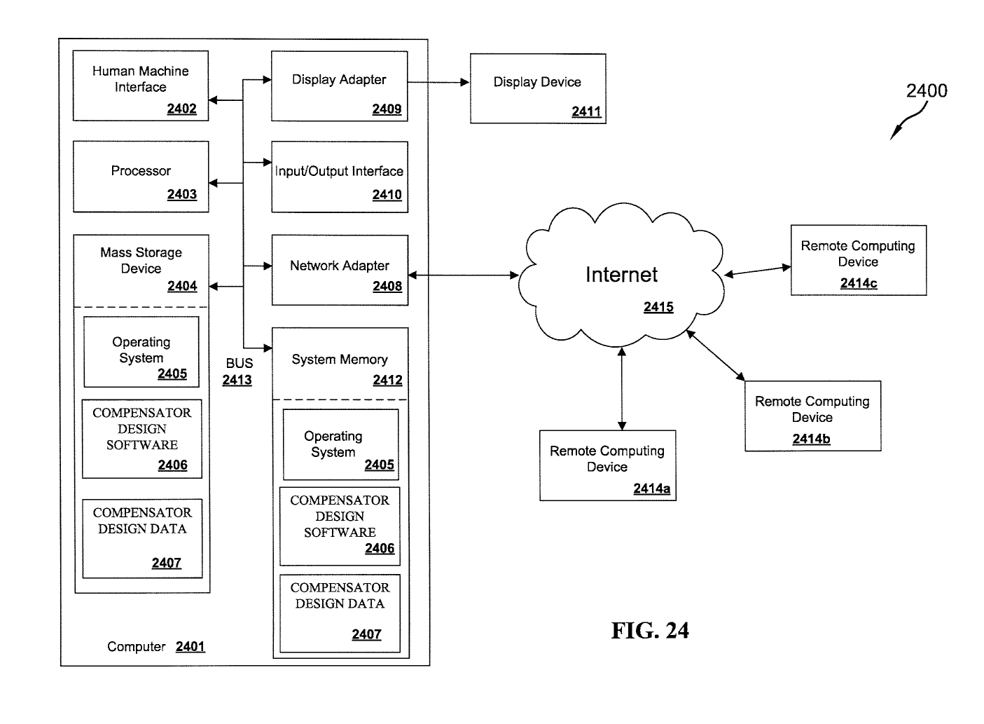

FIG. 24 illustrates a block diagram of an exemplary operating environment 2400 that enables various features of the subject disclosure and performance of the various methods disclosed herein. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The various embodiments of the subject disclosure can be operational with numerous other general purpose or special purpose computing system environments or configurations. Examples of well known computing systems, environments, and/or configurations that can be suitable for use with the systems and methods comprise, but are not limited to, personal computers, server computers, laptop devices or handheld devices, and multiprocessor systems. Additional examples comprise wearable devices, mobile devices, set top boxes, programmable consumer electronics, network PCs, minicomputers, mainframe computers, distributed computing environments that comprise any of the above systems or devices, and the like.

The processing effected in the disclosed systems and methods can be performed by software components. The disclosed systems and methods can be described in the general context of computer-executable instructions, such as program modules, being executed by one or more computers or other computing devices. Generally, program modules comprise computer code, routines, programs, objects, components, data structures, etc. that perform particular tasks or implement particular abstract data types. The disclosed methods also can be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 2401. The components of the computer 2401 can comprise, but are not limited to, one or more processors 2403, or processing units 2403, a system memory 2412, and a system bus 2413 that couples various system components including the processor 2403 to the system memory 2412. In the case of multiple processing units 2403, the system can utilize parallel computing.

In general, a processor 2403 or a processing unit 2403 refers to any computing processing unit or processing device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally or alternatively, a processor 2403 or processing unit 2403 can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Processors or processing units referred to herein can exploit nano-scale architectures such as, molecular and quantum-dot based transistors, switches and gates, in order to optimize space usage or enhance performance of the computing devices that can implement the various aspects of the subject disclosure. Processor 2403 or processing unit 2403 also can be implemented as a combination of computing processing units.

The system bus 2413 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 2413, and all buses specified in this description also can be implemented over a wired or wireless network connection and each of the subsystems, including the processor 2403, a mass storage device 2404, an operating system 2405, compensator design software 2406, compensator design data 2407, a network adapter 2408, system memory 2412, an Input/Output Interface 2410, a display adapter 2409, a display device 2411, and a human machine interface 2402, can be contained within one or more remote computing devices 2414a,b,c at physically separate locations, connected through buses of this form, in effect implementing a fully distributed system.

In one aspect, compensator design software 2406 can comprise computer-executable instructions for implementing the various methods described herein, such as exemplary method 2200. In another aspect, compensator design software 2406 can include software to control various aspects of manufacturing of the radiation compensator and, as part of manufacturing, treating a surface in accordance with aspects described herein in order to attain a desired thickness profile for the surface of the radiation compensator. In certain embodiments, compensator design software 2406 also can include computer-executable instruction for selecting radiopaque materials for manufacturing the radiation compensator. Compensator design software 2406 and compensator design data 2407 configure processor 2403 to perform the one or more steps of the methods described herein. In addition or in the alternative, compensator design software 2406 and compensator design data 2407 can configure processor 2403 to operate in accordance with various aspects of the subject disclosure.

The computer 2401 typically comprises a variety of computer readable media. Exemplary readable media can be any available media that is accessible by the computer 2401 and comprises, for example and not meant to be limiting, both volatile and non-volatile media, removable and non-removable media. The system memory 2412 comprises computer readable media in the form of volatile memory, such as random access memory (RAM), and/or non-volatile memory, such as read only memory (ROM). The system memory 2412 typically contains data and/or program modules such as operating system 2405 and compensator design software 2406 that are immediately accessible to and/or are presently operated on by the processing unit 2403. Operating system 2405 can comprise OSs such as Windows operating system, Unix, Linux, Symbian, Android, iOS, Chromium, and substantially any operating system for wireless computing devices or tethered computing devices.

In another aspect, the computer 2401 also can comprise other removable/non-removable, volatile/non-volatile computer storage media. By way of example, FIG. 24 illustrates a mass storage device 2404 which can provide non-volatile storage of computer code, computer readable instructions, data structures, program modules, and other data for the computer 2401. For example and not meant to be limiting, a mass storage device 2404 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 2404, including by way of example, an operating system 2405, and compensator design software 2406. Each of the operating system 2405 and compensator design software 2406 (or some combination thereof) can comprise elements of the programming and the compensator design software 2406. Data and code (e.g., computer-executable instruction(s)) can be retained as part of compensator design software 2406 and can be stored on the mass storage device 2404. Compensator design software 2406, and related data and code, can be stored in any of one or more databases known in the art. Examples of such databases comprise, DB2®, Microsoft® Access, Microsoft® SQL Server, Oracle®, mySQL, PostgreSQL, and the like. Further examples include membase databases and flat file databases. The databases can be centralized or distributed across multiple systems.

In another aspect, the user can enter commands and information into the computer 2401 via an input device (not shown). Examples of such input devices comprise, but are not limited to, a camera; a keyboard; a pointing device (e.g., a “mouse”); a microphone; a joystick; a scanner (e.g., barcode scanner); a reader device such as a radiofrequency identification (RFID) readers or magnetic stripe readers; gesture-based input devices such as tactile input devices (e.g., touch screens, gloves and other body coverings or wearable devices), speech recognition devices, or natural interfaces; and the like. These and other input devices can be connected to the processing unit 2403 via a human machine interface 2402 that is coupled to the system bus 2413, but can be connected by other interface and bus structures, such as a parallel port, game port, an IEEE 1394 Port (also known as a Firewire port), a serial port, or a universal serial bus (USB).

In yet another aspect, a display device 2411 also can be connected to the system bus 2413 via an interface, such as a display adapter 2409. It is contemplated that the computer 2401 can have more than one display adapter 2409 and the computer 2401 can have more than one display device 2411. For example, a display device can be a monitor, an LCD (Liquid Crystal Display), or a projector. In addition to the display device 2411, other output peripheral devices can comprise components such as speakers (not shown) and a printer (not shown) which can be connected to the computer 2401 via Input/Output Interface 2410. Any step and/or result of the methods can be output in any form to an output device. Such output can be any form of visual representation, including, but not limited to, textual, graphical, animation, audio, tactile, and the like.

The computer 2401 can operate in a networked environment using logical connections to one or more remote computing devices 2414a,b,c. By way of example, a remote computing device can be a personal computer, portable computer, a mobile telephone, a server, a router, a network computer, a peer device or other common network node, and so on. Logical connections between the computer 2401 and a remote computing device 2414a,b,c can be made via a local area network (LAN) and a general wide area network (WAN). Such network connections can be through a network adapter 2408. A network adapter 2408 can be implemented in both wired and wireless environments. Such networking environments are conventional and commonplace in offices, enterprise-wide computer networks, intranets, and the Internet. Networking environments are referred to as network(s) 2415 and generally can be embodied in wireline networks or wireless networks (e.g., cellular networks, such as Third Generation (3G) and Fourth Generation (4G) cellular networks, facility-based networks (femtocell, picocell, Wi-Fi networks, etc.).

As an illustration, application programs and other executable program components such as the operating system 2405 are illustrated herein as discrete blocks, although it is recognized that such programs and components reside at various times in different storage components of the computing device 2401, and are executed by the data processor(s) of the computer. An implementation of compensator design software 2406 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer-readable media can comprise “computer storage media,” or “computer-readable storage media,” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In various embodiments, the disclosed systems and methods for CBT can employ artificial intelligence (AI) techniques such as machine learning and iterative learning for identifying patient-specific, treatment-specific compensators. Examples of such techniques include, but are not limited to, expert systems, case based reasoning, Bayesian networks, behavior based AI, neural networks, fuzzy systems, evolutionary computation (e.g., genetic algorithms), swarm intelligence (e.g., ant algorithms), and hybrid intelligent systems (e.g., Expert inference rules generated through a neural network or production rules from statistical learning).

While the systems, devices, apparatuses, protocols, processes, and methods have been described in connection with exemplary embodiments and specific illustrations, it is not intended that the scope be limited to the particular embodiments set forth, as the embodiments herein are intended in all respects to be illustrative rather than restrictive.

Unless otherwise expressly stated, it is in no way intended that any protocol, procedure, process, or method set forth herein be construed as requiring that its acts or steps be performed in a specific order. Accordingly, in the subject specification, where description of a process or method does not actually recite an order to be followed by its acts or steps or it is not otherwise specifically recited in the claims or descriptions of the subject disclosure that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification or annexed drawings, or the like.

It will be apparent to those skilled in the art that various modifications and variations can be made in the subject disclosure without departing from the scope or spirit of the subject disclosure. Other embodiments of the subject disclosure will be apparent to those skilled in the art from consideration of the specification and practice of the subject disclosure as disclosed herein. It is intended that the specification and examples be considered as non-limiting illustrations only, with a true scope and spirit of the subject disclosure being indicated by the following claims.

What is claimed is:

1. A method, comprising:
receiving data indicative of a radiation treatment and topology of a region to be treated;
generating a position-dependent thickness profile of a radiation compensator surface based on the data indicative of the radiation treatment and the topology of the region to be treated; and
generating a plurality of dwell times for a radiation source based on the thickness profile, wherein the radiation source is movably coupled to a radiation compensator and is adapted to reside at a plurality of locations within the radiation compensator during a respective plurality of periods, each period of the plurality of periods being equal to a respective dwell time of the plurality of dwell times.

2. The method of claim 1, further comprising supplying a treatment plan comprising the position-dependent thickness profile and the plurality of dwell times.

3. The method of claim 1, wherein generating a position-dependent thickness profile of a radiation compensator surface based on the data indicative of the radiation treatment and the topology of the region to be treated comprises:
discretizing the radiation compensator surface into a plurality of voxels and assigning a respective initial plurality of thicknesses to the plurality of voxels; and
determining an extremum of an objective function by iteratively updating each thickness of the respective initial plurality of thicknesses and each dwell time of an initial plurality of dwell times, wherein the objective function is indicative of a difference among a prescribed dose at a position in the region to be treated and an actual dose provided at the position, the updating step yielding a current plurality of thicknesses and a current plurality of dwell times.

4. The method of claim 3, in response to identifying the extremum, performing the steps of:
configuring the current plurality of thicknesses as the position-dependent thickness profile; and
configuring the current plurality of dwell times as the plurality of dwell times.

5. The method of claim 1, further comprising providing a radiation compensator having a treated surface having a thickness according to the position-dependent thickness profile.

6. The method of claim 5, wherein providing the radiation compensator comprises etching a non-treated surface of the radiation compensator, wherein the non-treated surface is a substrate of a radiopaque material, the radiopaque material comprising at least one of a first high atomic-number material, a mixture of a plastic and a second high atomic-number material, and a mixture of a rubber and a third high atomic-number material.

7. The method of claim 6, wherein the etching step comprises removing the radiopaque material in an amount effective to yield the thickness profile.

8. The method of claim 5, wherein providing the radiation compensator comprises treating a non-treated surface of the radiation compensator with a radiopaque material, wherein the treating step yields the treated surface.

9. The method of claim 8, further comprising aligning the radiation compensator inside an applicator configured to implement at least part of the radiation treatment.

10. The method of claim 8, further comprising monitoring thickness of the treated surface in response to the treating step and at one or more locations in the treated surface.

11. The method of claim 8, wherein the non-treated surface of the radiation compensator comprises a substrate of a radiotransparent material, and wherein the treating step comprises printing ink onto the substrate in an amount effective to produce the thickness profile, the ink containing the radiopaque material.

12. The method of claim 8, wherein the non-treated surface of the radiation compensator comprises a substrate of a radiotransparent material, and wherein the treating step comprises etching the substrate according to the thickness profile, wherein the etching step yields an etched substrate.

13. The method of claim 8, wherein the treating step further comprises coating the etched substrate with a radiopaque material.

14. The method of claim 13, wherein treating step further comprises sintering at least a portion of the radiopaque material.

15. The method of claim 8, wherein the treating step comprises sputtering the non-treated surface of the radiation compensator with the radiopaque material.

16. The method of claim 8, wherein treating the non-treated surface of the radiation compensator comprises milling a portion of the radiopaque material according to a predetermined thickness profile.

17. The method of claim 16, wherein the milling step comprises cutting the portion of the radiopaque material in a sequence of rotations of said portion.

18. The method of claim 8, wherein treating the non-treated surface of the radiation compensator comprises:
milling at least one pocket in a slab of a solid material, the pocket having a depth determined by a specific thickness profile;
filling the at least one pocket with an amount of the radiopaque material; and
laminating the slab of solid material having the at least one pocket filled with the radiopaque material.

19. The method of claim 8, wherein the radiopaque material is a metal having an atomic number of at least 22.

20. The method of claim 5, further comprising providing a radiation delivery device comprising the radiation compensator.

21. A device, comprising:
a radiation compensator having a treated surface having a position-dependent thickness according to a thickness profile based on a radiation therapy plan and geometry of a region to be treated; and
a source of radiation movably inserted into a first enclosure coupled to the radiation compensator, wherein the radiation source is adapted to reside at a plurality of locations within the radiation compensator during a respective plurality of periods, each period of the plurality of periods being equal to a respective dwell time of the plurality of dwell times, and wherein each dwell time is based on the radiation therapy plan.

22. The device of claim 21, wherein the radiation compensator resides within a second enclosure that encompasses the first enclosure, the first enclosure adapted to move relative to the second enclosure, and wherein the second enclosure is coupled to alignment means for positioning the first enclosure relative to the second enclosure.

23. The device of claim 22, wherein the alignment means for positioning the first enclosure relative to the second enclosure comprises:
means for indicating orientation of the second enclosure relative to the region to be treated; and
means for locking at least part of the first enclosure outside the second enclosure in response to misalignment between orientation of the first enclosure and the orientation of the second enclosure.

24. The device of claim 23, wherein the means for indicating orientation of the second enclosure relative to the region to be treated are adapted to be visible on an three-dimensional imaging system.

25. The device of claim 24, wherein the first enclosure is a catheter and the source of radiation is movably inserted into the catheter via insertion means.

26. The device of claim 25, wherein the second enclosure is an applicator composed of a flexible biocompatible material.

27. The device of claim 26, wherein the radiation compensator resides outside the catheter.

28. The device of claim 27, wherein the radiation compensator resides within the catheter.

29. The device of claim 21, wherein the radiation compensator is coated with a radiopaque material.

30. The device of claim 29, wherein the radiopaque material is a metal having an atomic number of at least 22.

31. The device of claim 29, wherein the radiopaque material comprises one or more of barium, barium sulphate, bismuth, bismuth subcarbonate, tantalum, tin, silver, molybdenum, platinum, or titanium.

32. The device of claim 22, wherein the radiopaque material comprises one or more of a bismuth alloy, a tantalum alloy, a tin alloy, a silver allow, a molybdenum alloy, or a platinum alloy.

33. The device of claim 29, wherein the radiopaque material comprises lead.

34. The device of claim 29, wherein the radiopaque material further comprises one or more of lead powder or at least one etched lead sheet.

35. The device of claim 29, wherein the radiopaque material comprises gold.

36. The device of claim 29, wherein the radiopaque material further comprises gold nanoparticles.

37. The device of claim 29, wherein the radiopaque material comprises tungsten.

38. The device of claim 37, wherein the radiopaque material further comprises tungsten powder.

39. The device of claim 29, wherein the radiopaque material comprises iron.

40. The device of claim 39, wherein the radiopaque material further comprises one or more of iron powder or iron nanoparticles.
